(19) United States
(12) Patent Application Publication
(10) Pub. No.: US 2026/0174932 A1
(43) Pub. Date: Jun. 25, 2026

(54) METHODS AND COMPOSITIONS FOR PARTICULATED AND RECONSTITUTED TISSUES

(71) Applicant: The Regents of the University of Colorado, a body corporate, Denver, CO (US)

(72) Inventors: Corey Neu, Boulder, CO (US); Jeanne Barthold, Fort Collins, CO (US)

(73) Assignee: The Regents of the University of Colorado, a body corporate, Denver, CO (US)

(21) Appl. No.: 19/537,885

(22) Filed: Feb. 12, 2026

Related U.S. Application Data
(62) Division of application No. 16/647,488, now abandoned.
(60) Provisional application No. 62/559,268, filed on Sep. 15, 2017.

(51) Int. Cl. A61L 27/44; A61L 27/36

(52) U.S. Cl. CPC A61L 27/44; A61L 27/3612; A61L 27/362; A61L 27/3654; A61L 27/3675; A61L 27/3683

(57) ABSTRACT

Particulated and reconstituted tissues comprising small, densely packed tissue microparticles encapsulated in a tissue specific promoting gel packed at a percolation threshold that can be transplanted into damaged tissue thereby facilitating regeneration following trauma to the tissue. The engineered microparticle construct for tissue replacement and repair, as taught herein, provides numerous benefits including (1) encouraging a regenerative response in damaged tissue regions, (2) mimicking the structural support of native tissue, (3) establishing an environment that promotes attachment, migration, and differentiation of infiltrating stem cells, and (4) providing a source of growth factors and other anti-catabolic growth factors and cytokines. Tissue specific microparticles packed together at, or past, their percolation threshold will provide the necessary mechanical environment and to best recapitulate and integrate with native tissue. The packing of microparticles, derived from the ECM of native tissue to a concentration past the percolation point will yield both the necessary biochemical and biomechanical properties necessary for reconstituting a specific tissue.

### CROSS-REFERENCE TO RELATED APPLICATIONS

This application is a divisional application of U.S. patent application Ser. No. 16/647,488, filed Mar. 14, 2020, which was the National Stage Entry of International Application No. PCT/US18/51355, filed Sep. 17, 2018, which claims the benefit of U.S. Provisional Application No. 62/559,268, filed Sep. 15, 2017.

### STATEMENT OF GOVERNMENT INTEREST

This invention was made with government support under grant numbers AR063712, AR066230, AR064178, awarded by the National Institutes of Health, and grant number CMMI1349735 awarded by the National Science Foundation. The government has certain rights in the invention.

### FIELD OF INVENTION

This invention relates to tissue healing and repair. More specifically, this invention relates to tissue microparticles encapsulated in a tissue specific promoting gel that can be transplanted into damaged tissue thereby facilitating regeneration following trauma to the tissue.

### BACKGROUND OF THE INVENTION

Osteoarthritis (OA) is a debilitating disease that affects nearly 20% of the U.S. population. Decellularized cartilage, which contains a mature, healthy extracellular matrix (ECM), has been explored as a tissue replacement strategy for OA. The extracellular matrix (“ECM”) transmits biomechanical signals from outside of the construct to the cells to initiate critical cartilage-specific signaling cascades via cell-ECM interactions with collagens, glycoproteins, and proteoglycans that are conserved in a de-cellularized three-dimensional ECM. Unfortunately, large decellularized cartilage allografts suffer from poor implant diffusion characteristics and cellular infiltration. Decellularized cartilage microparticles allow for improved cell infiltration, but they commonly utilize severe chemical agents that adversely degrade matrix proteins and affect cell differentiation and fail to attain clinically relevant mechanical properties required for implant survival.

Cartilage, muscle, and skin are three very different tissue types that all perform important tasks in the human body. Loss of function for any of these tissues carries foreboding consequences for the human body. Cartilage tissue is highly organized, and is composed primarily of crosslinked collagen II, proteoglycans, and water, which together provide joint lubrication and facilitate load transmission during normal movement. The loss of cartilage due to disease or overuse leads to an inability for the remaining tissue to compress or lubricate joints properly when exposed to mechanical loading. The degenerative joint condition known as osteoarthritis causes cartilage tissue to become softer and more unstructured, which is then increasingly susceptible to continuous breakdown and loss of function of this important tissue. Moreover, skin tissue is rich in collagen I, whose high elasticity is important for providing protection for your organs while being flexible in everyday movements. Skin tissue makes up the largest organ in the body and serves as human body's main protective barrier from external forces and organisms. In the case of massive skin tissue injuries such as large burns or deep wounds, skin regeneration is impossible without the use of exogenous materials, leading to high rates of infection and sometimes death. Finally, skeletal muscle is composed mainly of collagens and proteoglycans that interact in a unique way to give muscle is contractile abilities. The loss of large areas of muscle from either traumatic or surgical events, termed volumetric muscle loss, leads to an inability for muscle recovery, and major loss of function. While these three tissues represent diverse tissue structures and biomolecular compositions, all three have a molecular architecture that endows the tissue with important functional properties, and when lost can lead to serious clinical outcomes.

### SUMMARY OF THE INVENTION

The present invention provides particulated and reconstituted tissues comprising small, densely packed tissue microparticles encapsulated in a tissue specific promoting resin/gel that can be transplanted into damaged tissue thereby facilitating regeneration following trauma to the tissue. The engineered microparticle construct of the invention, as taught herein, provides numerous benefits including (1) encouraging a regenerative response in damaged tissue regions, (2) mimicking the structural support of native tissue, (3) establishing an environment that promotes attachment, migration, and differentiation of infiltrating stem cells, and (4) providing a source of growth factors and other anti-catabolic growth factors and cytokines. Tissue specific microparticles packed together at, or past, their percolation threshold will provide the necessary mechanical environment and to best recapitulate and integrate with native tissue. The packing of microparticles, derived from the ECM of native tissue to a concentration past the percolation point will yield both the necessary biochemical and biomechanical properties necessary for reconstituting a specific tissue.

The present invention provides tailored and engineered biomaterials, using native tissues, that closely mimic and recreate tissues of the body. The process for producing the biomaterials of the invention includes breaking a healthy tissue into very small (micron scale, i.e. micronized) fragments or tissue particles, processing the tissue particles to remove cellular debris, and then recombining the tissue particles utilizing a resin to aggregate the particles and enable the particles to fill a tissue defect. The recombination of components into a tissue can therefore include: (1) tissue particles, (2) a resin that encapsulates the particles, and (3) the addition of cell sources and soluble growth factors.

The native, healthy tissue may be xenogeneic, allogeneic, autogenic, or syngeneic, depending upon the needs and circumstances of the application. The tissue can be harvested from any tissue that is to be regenerated, including cartilage, skin, ligament, meniscus, tendon, muscle, heart, brain, lung. By way of example, where one desires to regenerate cartilage, one would start with a healthy cartilage sample. The harvested sample would then be particulated into sub-millimeter to micron scale to yield tissue particles.

The tissue particles can then be decellularized and/or devitalized. As discussed above, the tissue particles will be selected to match to tissue to be generated to drive tissue type. The tissue particles will also be matched in size to drive both the mechanical properties of the desired regenerated tissue and to drive cell differentiation and gene expression.

The resulting tissue particles can then be placed in a resin material. The resin material can be moldable, starting as a liquid and hardened with heat. In this manner, the resin material and tissue particle mixture can be formed into any shape to fill a tissue defect. The resin could be comprised of hyaluronic acid, collagen, fibrin, chondroitin sulfate, heparin, while the decellularized matrix could comprise cartilage, adipose, or other tissues. Oligomeric collagen I can be cross linked into a gel and the properties can be adjusted properties via densification. By varying concentration HA/PEGDA you can vary the pore size and stiffness of the resulting gel. Adipose tissue has been used previously to make gels in 3D printing applications, while collagen cross-links naturally. Adipose and collagen are particularly suited for application in more fat-based tissues. Agarose is a highly tunable gel when used in varying concentrations. Fibrin is a current hospital standard and works as a resin for the present application. Polymers could be substances such as PEG, PGLA, peptides.

Resins can be employed to encapsulate tissue particles such that the resulting composition creates a tissue mimic for any tissue type (e.g. cartilage, skin, ligament, meniscus, tendon, muscle, heart, brain, lung). Resins could be comprised of collagen (e.g. types I or II), fibrin, agarose, hyaluronic acid (HA), HA/PEGDA (PEG diacrylate M.W 3400 crosslinked with HA that has been functionalized with thiol groups), decellularized adipose tissue, PEGDA crosslinked with UV. They can be used to create a positive, tunable cellular environment that allow for transplantation in vivo. The resins can be chosen to be cross-linkable and densifiable, with controllable porosity for nutrient flow and delivery. The tissue particle-resins composition can be layerable, with distinct layers that mimic complex tissue environments, or it can be packable, with mechanical rigor to withstand in vivo loading. The tissue particle-resins composition can be combined with other soluble factors, such as growth factors, cytokines, peptidoglycans, anti-inflammatory agents, anti-senescent agents, and cross-linking agents. The resins can encapsulate matter from xenogenic, allogenic, autogenic, syngeneic cells sources, including primary cells, stem cells, progenitor cells, engineered cells, and other altered/transformed/immortalized cells. Particles, resin, cell sources, and soluble factors can be combined and delivered in vivo, using common or custom-made injection/delivery systems.

The methods and compositions of the invention will have an immediate and long-term impact on patient care and restoration of function for those personnel who have sustained traumatic orthopedic injuries. The present invention provides the musculoskeletal and pharmaceutical communities with (i) new engineered materials with the potential for transformative cartilage repair and for the repair of a wide variety of other medically important tissues, (ii) baseline data describing cell signaling and biomechanics at the subcellular level, (iii) the ability to functionally evaluate the efficacy of emerging biological therapies for defect repair and OA, and (iv) a platform technology to more broadly study mechanical function and repair of other load-bearing tissues (e.g. ligament, skin).

### BRIEF DESCRIPTION OF THE DRAWINGS

For a fuller understanding of the invention, reference should be made to the following detailed description, taken in connection with the accompanying drawings, in which:

FIG. 1 is set of diagrams, images, and graphs depicting that the engineered microparticle construct is a versatile tissue engineering platform that utilizes decellularized tissue in a biocompatible gel to mimic native tissue environments. (A) Illustration of the protocol used to produce the engineered microparticle construct for tissue repair. (B) Microscopic images of native cartilage tissue, decellularized cartilage, and decellularized microparticles at two densities, with particles (white arrow) and void spaces (light arrowhead) visualized (DAPI stain, 405 laser, confocal z-stack). (C). White light macroscopic and 405 laser microscopic images of the three different tissues reveal the differences between native tissue, decellularized tissue, and decellularized microparticles for each tissue type. (D). Raman spectroscopy spectra for each decellularized tissue type with annotated peaks of common collagen protein signatures.

FIG. 2 is set of images and graphs depicting increased particle density in gels beyond a percolation threshold that dramatically increased compressive modulus. (A) Increasing the weight of particles also increased the area ratio in the composite gels until a point. After that point, additional compression was necessary to achieve higher area ratios (centrifugation). (B) Gels were mechanically tested at 0.1%/sec to avoid stiffening effects of water in the hydrogels to a deformation of 40%. Compressive modulus was calculated from 30% to 40% strain, at the linear portion of the stress vs. strain curve. (D) When plotted against area ratio, the modulus plot has a clear inflection point at 0.55 area ratio. When a percolation model was fit to this data, known as the General Effective Medium (GEM) model, it confirms that the percolation threshold of the data lies at 0.57 area ratio. (C) Furthermore, when gels of different crosslinking percentages and different particles sizes were tested under physiological conditions of a typical step (compression to 20% in 50 ms and held at 20% for 30 min) it was found that gels with particles behaved viscoelastically, and modulus was much closer to native cartilage than measured in just gel constructs.

FIG. 3 is a pair of drawings and a set of images depicting the size controlled microparticles that are evenly distributed throughout the height of the polymerized gel structure. (A) Schematic for gel slicing and imaging. Polymerized HA/PEGDA gels were cut down the middle and were imaged from the top to bottom of the gel to visualize particle size and distribution throughout the gel. (B) Gels were stained using Ghost Die 710 and imaged on a confocal microscope (Nikon) with a 10× objective lens.

FIG. 4 is a set of images addressing the percolation threshold of particles in HA/PEGDA gels. (A) Gels are made with different ratios of particle weight: gel volume. After polymerization, the gels are extracted from mold and placed in 35 mm petri dishes. (B-D) PBS is then introduced to the area surrounding the gels, and the dishes are gently swirled around to dislodge any particles that are not polymerized with the gel. (B) The smallest ratio of 0.28 g/L and (C) the medium ratio of 0.24 g/L had loose particles after polymerization, while the final ratio of 0.22 g/L (D) was enough to encapsulate all the particles into the gel. All tests performed using 250 μm particles and 1% HA/PEGDA gel configuration. Smaller particle sizes may promote positive cell responses, while larger particle sizes may improve mechanical properties. Particle sizes range from 1-5000 μm, though commonly are size sorted for 90 μm-250 μm. HA/PEGDA gel configurations range from 0.5%-3%.

FIG. 5 is a series of images showing an exemplary methodology for creating particulated and reconstituted tissues. In one aspect the invention provides a composite material of decellularized tissue microparticles in a resin. The composite material can promote cellular infiltration, construct integrity, and mechanobiology. (A) One example of an application of the invention is a tissue defect injury that the compositions and methods of the invention can be used to fill is a medial condyle defect in cartilage. The defect is filled with size controlled microparticles and gel resin. (B) Tissue that is type-specific to the injury space is harvested from xenogenic, allogenic, autogenic, or syngenic source. Tissue is devitalized and pulverized in a liquid nitrogen freezer mill and size controlled for the formation of microparticles. Particles are then decellularized in 1-3% SDS for 6-30 hours. (C) Target resin (e.g. HA/PEGDA, collagen, etc.) is created in liquid form and applied to a microparticle suspension filling the desired defect space. The microparticle and resin composite is heat polymerized to form a stiff gel.

FIG. 6 is a pair of drawings and a set of images depicting the ability of tissue microparticles and resin material to fill any shape tissue defect, as shown with this cartilage defect model filled with porcine cartilage microparticles and HA/PEGDA gel. Cartilage plugs with a cylindrical defect were treated with four different treatments: whole cartilage plug, no fill, PEGDA/HA Gel, and pulverized particles (by nitrogen pulverization, mortar and pestle) in a PEGDA/HA gel. (A) After staining with ghost die 710 (Tonbo Biosciences), each plug was cut in the middle and imaged in the 640 nm channel on a confocal microscope (Nikon Instruments). (B) Macroscopic and confocal microscopic images of the four different plug treatments.

FIG. 7 is a schematic of the invention injection system. A desired tissue defect will first be filled with tissue specific microparticles and then filled with the polymerizable resin chosen for the desired tissue type. (e.g. Collagen I and adipose both appropriate for collagen I heavy tissues-skin, breast implant, etc.) Injection will allow a precise, press fit fill of tissue microparticles and resin into the tissue defect.

FIG. 8 is a set of four images addressing microparticle and HA/PEGDA resin composite in an ovine knee joint to show the feasibility of gel polymerization in a tissue defect. A knee joint was dissected to expose the condyles (A). A defect is made in the medial condyle to the subchondral bone (B). The defect is filled with 250 μm microparticles and 1% HA/PEGDA gel composite (C). Viscous gel composite polymerizes and hardens into a gel inside of the defect (D).

FIG. 9 is a set of three images showing that microparticles can be encapsulated in many types of resin, depending on the target tissue. Shown here, microparticles are encapsulated 25 in both HA/PEGDA and Collagen. Confocal Imaging (A), H&E (B) and Safranin-O (C) all show the ability for microparticles to be encapsulated into a polymerized resin that can support cell infiltration and differentiation.

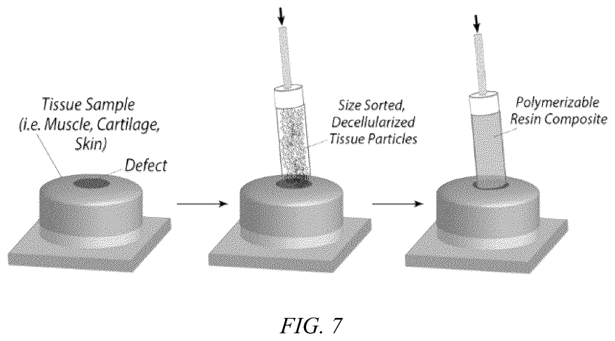

FIG. 10 is a set of three graphs showing that the engineered microparticle platform can be used to encapsulate particles in many resins (i.e. HA/PEGDA, agarose, fibrin glue). (A) HA/PEGDA with encapsulated 250 μm particles is the stiffness, most viscoelastic of all materials. (B) 1% low melt agarose is plotted versus agarose with 250 μm cartilage particles. (C) Fibrin glue gels are plotted against fibrin glue with encapsulated microparticles. All resin materials are strengthened compressively by the addition of cartilage microparticles.

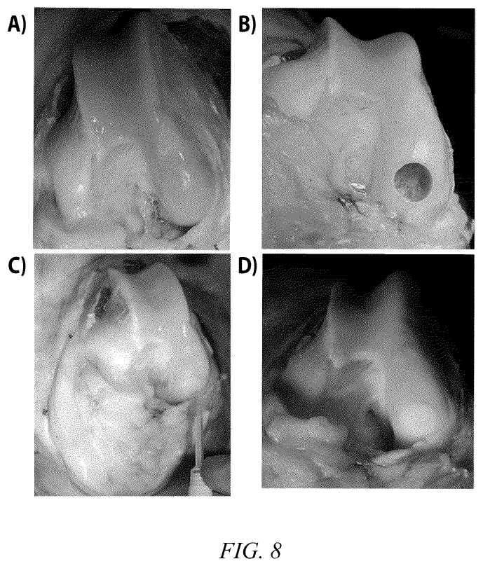

FIG. 11 is a set of two images and three graphs depicting the mechanical testing protocol for HA/PEGDA gels. (A) Unconfined compression testing was performed on a Bose ElectroForce 5500 mechanical testing system. Contact with gels was ensured using a 0.05N pre-load, followed by 20% compression of the gel in 50 ms. The platen was held at 20% compression for 30 minutes to evaluate equilibrium modulus. (B) An example testing curve in a control, medial cartilage plug. The plug is loaded to ˜25 N and then relaxes to ˜3 N in the remaining half hour. (C) Example testing curve of the 1% gel. The curve is noticeably different than cartilage, as it does not relax and behaves much more elastically. (D) Finally, the 1% gel with micro-particles encapsulated shows a similar type of curve to the cartilage, showing that the combination of particles and gel behave more like the cartilage than like the gel, but with lower mechanical properties.

FIG. 12 is a graph depicting that encapsulation of microparticles in gel resin allows for tunable mechanical properties with the aim to replicate the mechanics of the target tissue. Here, the instantaneous and equilibrium moduli were calculated for differing percentages of HA/PEGDA gels encapsulating no particles, 125 μm particles, and 250 μm particles (n=3-6). Gels were compared with the industry standard material for cartilage defect filler, fibrin glue. Mechanical testing of each resin particle combination is critical to match the tissue type. As seen above, there is an ideal composition depending on particle size and viscosity of the resin that allows for enhanced polymerization and therefore increased mechanical properties.

FIG. 13 is a set of two images demonstrating proof-of-concept repair of chondral repair by the engineered microparticle construct for cartilage repair using a sheep defect model in the femoral condyle of the knee. Using cadaveric tissues, the efficacy of microparticle-HA/PEGDA constructs was tested for preclinical studies involving critical sized (10 mm) defects representing tissue trauma (A, white arrowhead). These initial studies standardize surgical approaches and demonstrate that the engineered microparticle construct may be effectively transplanted for the repair of defects (B, black arrow).

FIG. 14 is a graph depicting the decellularization of skin tissue. Decellularization efficacy can be measured by the DNA content in the tissue after digestion and extraction of DNA.

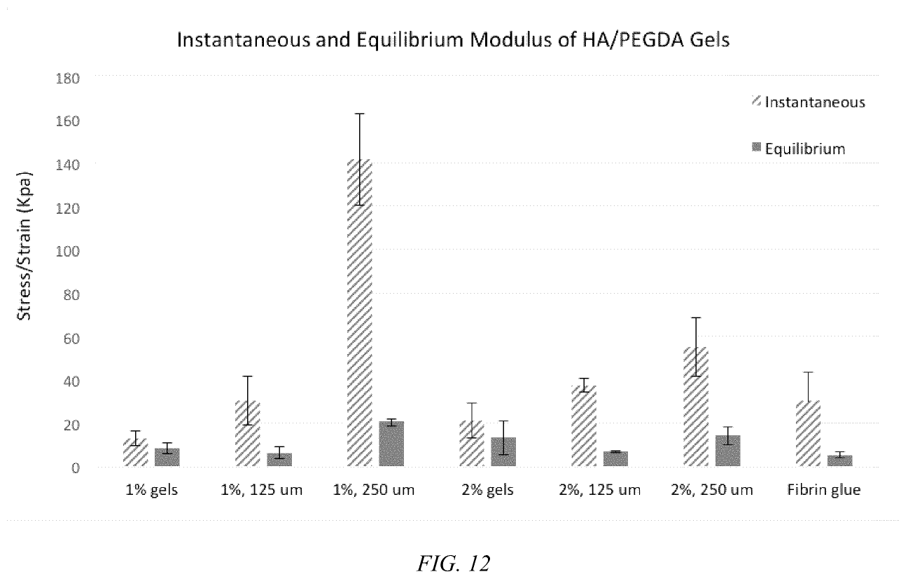

FIG. 15 is set of three graphs and an image depicting the mechanical and structural properties of the engineered microparticle construct for skin repair. Increased particle packing leads to higher area fraction in gels, which in turn leads to higher compressive modulus. The modulus of the 0.84 area ratio gels approaches that of decellularized native skin (A). All gels show a large amount of swelling after polymerization when they are introduced to PBS buffer (B). Gels swell more radially, than vertically. However, skin does not lose all structural properties in the decellularization, shown by the number of collagenous peaks in raman spectroscopy (C).

FIG. 16 is set of six images depicting mouse skin fibroblasts encapsulated in 3D hydrogels. eGFP tagged mouse fibroblasts were cultured on tissue culture plastic, in a hyaluronic acid-based gel, and in a HA based gel with porcine skin microparticles. Fibroblasts display a spherical phenotype in 3D, as compared to the extensions seen when cultured on tissue culture plastic. When cultured with the particles, cells are not spread evenly throughout the gel and seem to group around the particle edges, suggesting some communication between cell and particle.

FIG. 17 is set of images and a graph depicting chondrocytes introduced to the gel region of the composite recellularize microparticles. Cartilage gel constructs were imaged each day for a two-week culture (C). Using a thresholding technique to create an image mask (A), it was possible to quantify how many CFSE stained cells were present within the particles each day. (b) It was observed that the majority of cell migration from the gel phase into the particles happens in the first 2 days of culture. Scale bar=100 μm.

FIG. 18 is a pair of images, a diagram, and a histogram depicting the calculation of the area ratio using an overexpressed DAPI stain (A) to outline the particles and differentiate from the gel. The particles are then outlined (B) and an area is calculated both within and outside of the particle outline. When area ratio was calculated for the top of the gel, and the cross section (B), there was not a dramatic difference between the two area ratios calculated (D). Therefore, area ratio was calculated from the top of the gel.

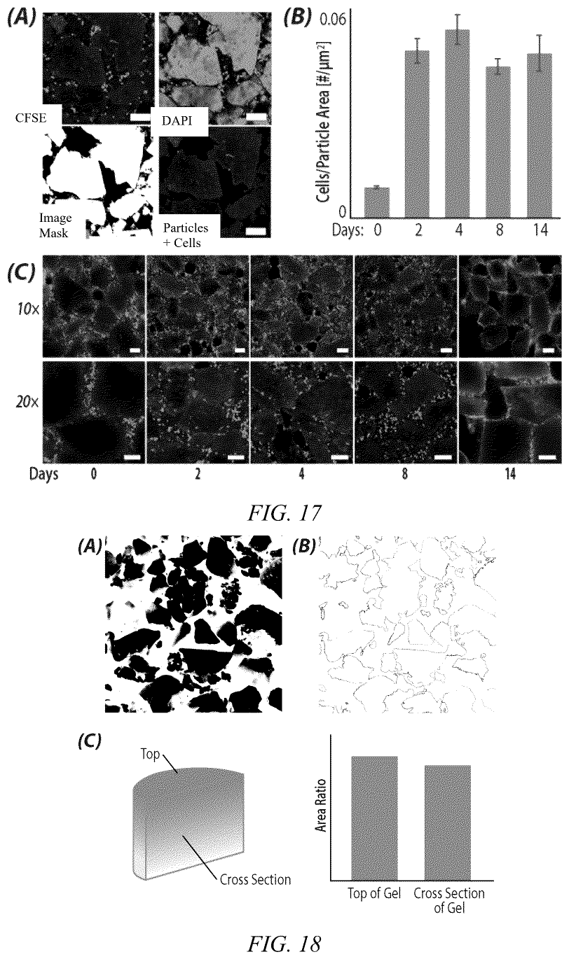

FIG. 19 is a pair of images and a histogram depicting that recellularization happens globally in the gel composite within the first 12 hours of culture and encourages chondrogenic gene expression. (A) On a global scale across the gel surface (scale=1000 μm), recellularization in the particles can be observed everywhere. (b) Using live imaging, recellularization can be observed in the first 12 hours of culture. (c) Gene expression shows that cells in the presence of particles at percolation threshold or beyond, show increased chondrogenic markers as compared to those cultured in HA/PEGDA gel alone. In particular, the upregulation of Sox9 and the down regulation of ColI suggests a limiting a fibrotic healing response.

### DETAILED DESCRIPTION OF THE PREFERRED EMBODIMENT

Microparticle-collagen constructs have been developed that combine the advantages of decellularized cartilage with the tunable features of densified collagen. The engineered microparticle construct of the invention provides a composite of decellularized tissue in a protein or biosynthetic gel resin, that exceeds the percolation threshold of microparticles, exhibits clinically relevant mechanical properties, and promotes proper differentiation of infiltrated human mesenchymal stem cells (hMSCs). Microparticle-collagen constructs with cartilage particles indicate that hMSCs in contact with, but not between, cartilage microparticles express chondrogenic markers, suggesting that dense packing of tissue microparticles is required. Furthermore, collagen is a protein gel resin that is tunable via densification but may be a better resin for collagen I based tissues such as skin, rather than cartilage, a very type II collagen rich tissue. Alternatively, hyaluronic acid (HA) is an important cartilage ECM glycosaminoglycan that interacts with cells via CD44 surface receptors to facilitate cell migration and differentiation. Hyaluronic acid interacts with aggrecan via cartilage link proteins, creating aggregates in cartilage tissue that significantly contribute to the mechanical strength of the tissue. Hyaluronic acid can be functionalized with thiol groups (following replacement of HA carboxyl groups) to react with poly(ethylene) glycol diacrylate (PEGDA) in a thiol-mediated Michael addition reaction. This cross-linking reaction modifies the linear molecule to form a stable 3D scaffold network that has tunable mechanics, porosity, and degradation rates. Therefore, both collagen and hyaluronic acid are examples of resins that can encapsulate tissue microparticles to initiate tissue specific differentiation responses of host stem cells. Furthermore, studies in our lab have shown that adipose ECM can also be broken down and formed into a gel to facilitate further tissue types. The resin of the engineered microparticle construct is vitally important for the signaling mechanisms and attachment sites for cells between the particles, and therefore the tissue target will dictate the specific type of resin to be utilized.

There are significant challenges in prior viable tissue microparticle systems, including limited donor availability, limited shelf life, and the possibility for disease transmission. The present technology, through the use of decellularized particulate tissues packed into a differentiation promoting resin, provides several key advantages over existing technologies including (1) the use of native extracellular matrix that can be derived from xenogenic, allogenic, autogenic, or syngeneic sources; (2) the ability to incorporate autologous cells from the patient that infiltrate naturally into the reconstituted matrix from underlying bone marrow; and (3) the inclusion of composites made from matrices (e.g. hyaluronan) that facilitate tissue specific differentiation and allow for a moldable, scalable, and mechanically tunable final tissue construct suitable for treatment of a wide variety of trauma (i.e. orthopedic, skin) presented in the clinic.

Small, densely packed tissue microparticles encapsulated in a tissue specific promoting gel transplanted to the damaged tissue will encourage regeneration following trauma. The engineered microparticle construct of the invention will: (1) encourage a regenerative response in damaged tissue regions; (2) mimic the structural support of native tissue; (3) establish an environment that promotes attachment, migration, and differentiation of infiltrating stem cells; and (4) provide a source of growth factors and other anti-catabolic growth factors and cytokines.

The present invention provides methods and compositions for the engineering of tissues of the body. The invention is presented in the context of three important aspects and/or components: (1) Tissue specific microparticles that are generated, processed (decellularized, devitalized), size-sorted, and packed into a tissue defect to fill the injury/defect space. (2) A resin (e.g. gel) that is mixed fresh and combined with the particles in the defect. The resin will fill in the space between particles and percolate through the gel to provide structural support for particles. (3) Autologous stem cells that enter the construct from the patient's own body (e.g. blood, adipose, bone marrow), depending on location, or addition of soluble factors exogenously or from the patient's own body. Stem cells will attach to the construct and will differentiate into a tissue specific lineage. The construct will polymerize with time, so no additional polymerization methodology (e.g. UV light) is required.

Although applicable to a variety of tissues in the body (e.g. spinal cord (discussed below), skin, muscle), initial efforts were motivated by and directed toward treatment of damaged or diseased articular cartilage. The first examples provided below address engineered microparticle constructs for cartilage repair. The current standard of care for a critical sized defect in cartilage is microfracture, a procedure where physicians drill into the marrow and let the blood initiate a healing response. This strategy is highly unsuccessful at relieving pain in a patient because the healing response leaves a highly collagen I dominated fibrous scar in the defect. This kind of healing repair generally leads to further degradation in the knee, as it is a mechanically- and biologically inferior replacement in an otherwise normal joint capsule. Other common approaches to fix a critical size defect in the knee include osteochondral allografts or autografts, matrix assisted autologous chondrocyte implantation (MACI), and DeNovo NT tissue graft. With an osteochondral autograft, a surgeon removes a piece of cartilage from a non-load bearing surface and implants the tissue into the defect. This procedure can often lead to donor site morbidity and is more susceptible to infection because it is an open joint, rather than arthroscopic, surgery. The only difference with an osteochondral allograft is the tissue is taken from a donor patient and used to fill the defect. The drawbacks of the allograft are the risk of disease transmission, and the limiting need for a donor.

MACI is a cellular based approach that consists of two surgeries. The first surgery involves extracting chondrocytes from a subject's cartilage. The cells are then grown in the lab and injected into the defect with a second surgical procedure. Finally, the DeNovo NT tissue graft is a cellularized tissue particle approach where donor tissue is cut into cm³ particles and filled into the defect with fibrin glue. This has similar drawbacks to the allografts because it relies on juvenile donor tissue (very limited source) and has a very short and expensive shelf life due to its cellular nature. While these solutions are the standard of care, none work very effectively at regenerating hyaline cartilage, and all have significant drawbacks.

One advantage to the present invention is that the microparticles are in the resin at their percolation threshold, and therefore cells have many tissue specific attachment sites. Furthermore, the resin is carefully chosen to match the tissue type so that the cells not in contact with the particles still have signaling cues similar to that of the tissue. For example, hyaluronic acid (HA) is chosen as a base for cartilage repair because it is associated with growth and regeneration in cartilage. HA has been shown to enhance attachment, migration, and chondrogenic differentiation. Additionally, human spinal cord is composed of similar components to hyaline cartilage and therefore HA resin can be used also for regenerating spinal cord (see Example 4, below). Alternatively, collagen I is an ideal resin to use for skin regeneration applications because it is an extremely prevalent protein in skin. Additionally, decellularized adipose matrix is an ideal resin to use for skin regeneration of breast reconstruction because of the high content of collagen and/or adipose.

Furthermore, our technology does not suffer from weaknesses of the above techniques seen in cartilage repair because the source of the tissue can be allogenic, autogenic, xenogenic, etc. Additionally, since the tissue particles are decellularized, they can have a much longer shelf life, are not dependent on a viable donor, will not have donor site morbidity complications, and have decreased risk of disease transmission. As a result, this scaffold is less expensive, readily available, and less risky to the patient than current technologies and techniques.

### Example 1—Engineered Microparticle Construct for Particulated and Reconstituted Cartilage for Cartilage Replacement and Repair—Overview

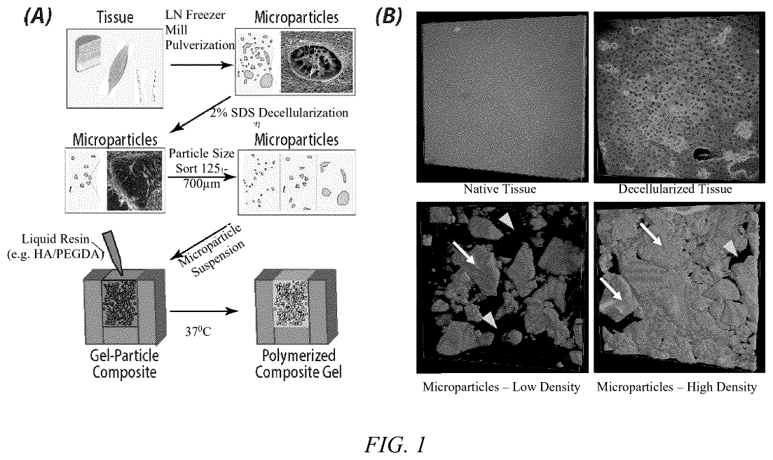

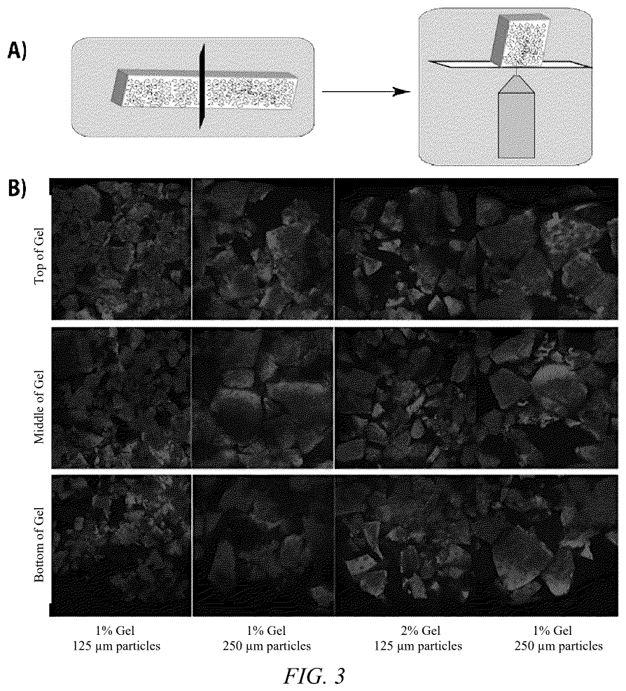

The present invention provides methods and compositions for particulated and reconstituted tissues as shown by way of example in FIGS. 1 and 5. The methods include the steps of pulverization, decellularizing, and size sorting specific tissue microparticles. These particles are then applied to a defect of their particular tissue type and reinforced with a polymerizable resin that mixes with native stem cells and natural growth factors in the recipient's blood or bone marrow.

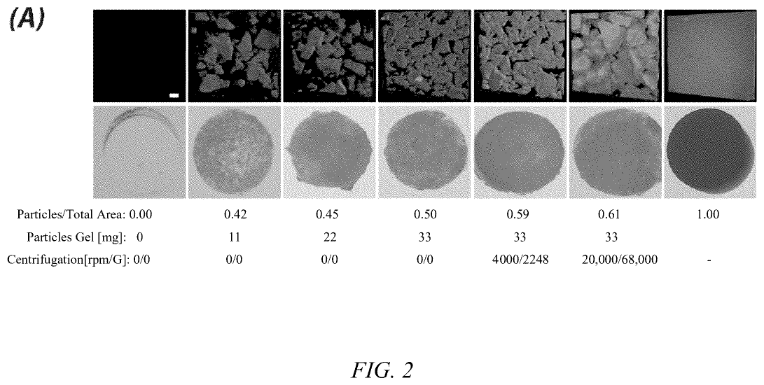

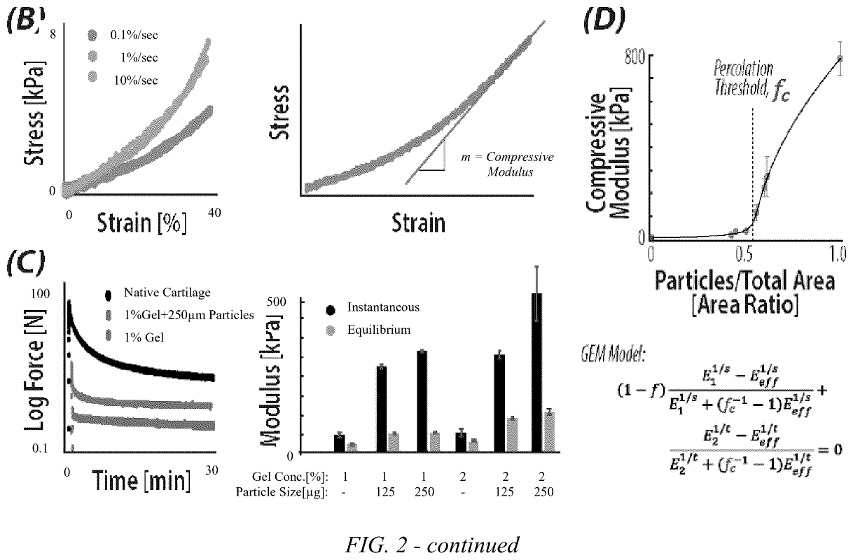

The methods taught herein enable the growth of scaffold-reinforced microparticle tissues for regenerative applications. Tissue specific particles (e.g. cartilage, skin, muscle, spinal cord) are encapsulated in a resin at their percolation threshold, with the minimum amount of resin applied as possible to simply to hold together the scaffold (FIG. 4). As a result, small tissue microparticles are tightly packed which maximize contact between the cells and microparticles (FIG. 3).

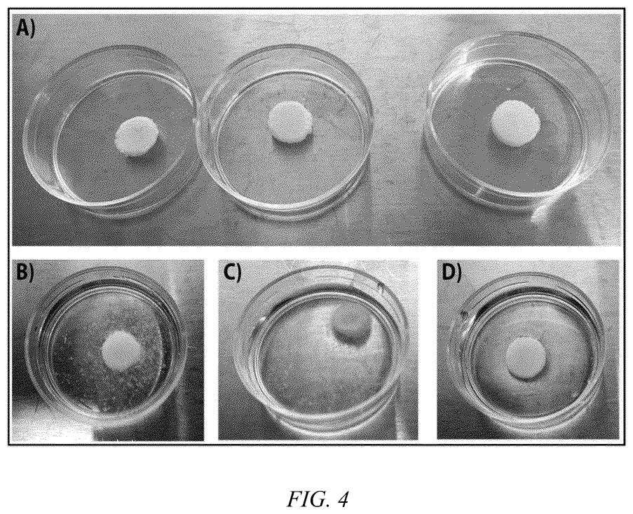

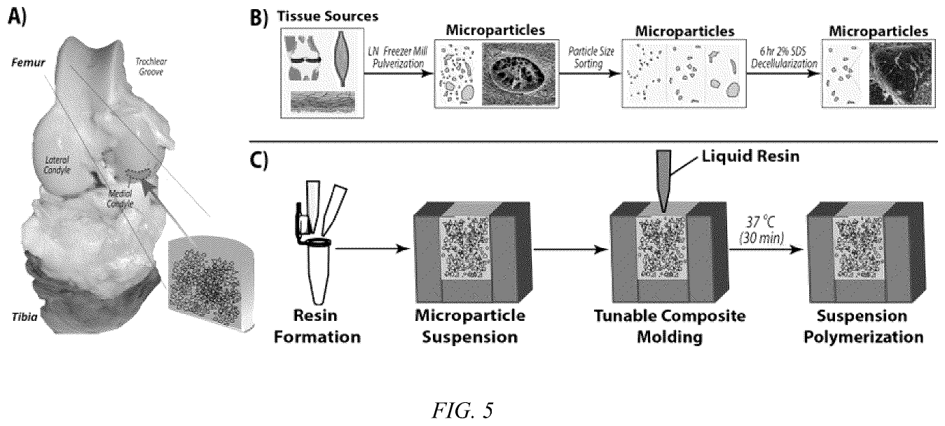

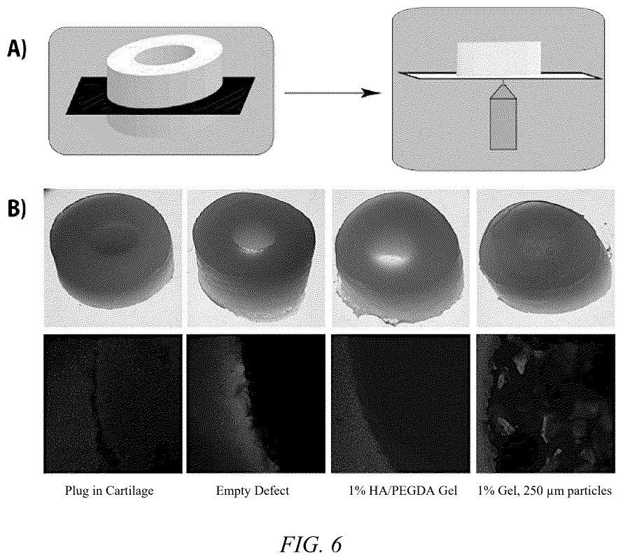

The technology taught herein is moldable to fit any size or shape of defect. The moldable properties and aspects of the technology are demonstrated herein in both in vivo applications using a sheep joint (FIG. 8) and in vitro applications using bovine cartilage plugs (FIG. 6). The application of the technology to spinal cord, skin, or muscle would be analogous to the applications demonstrated for cartilage. Specifically, the resin would be prepared and using a custom injection system (FIG. 7), tissue specific microparticles would be applied to the wound or defect, followed immediately with the polymerizable resin.

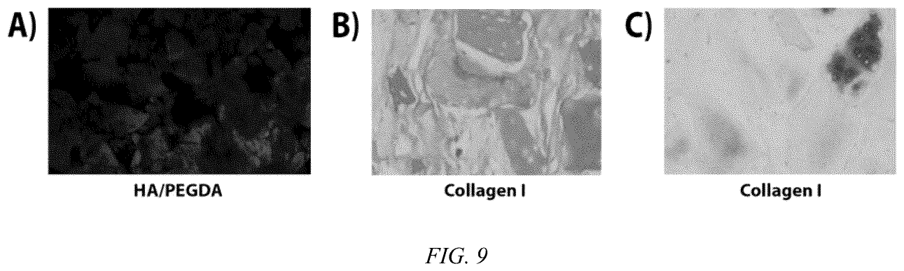

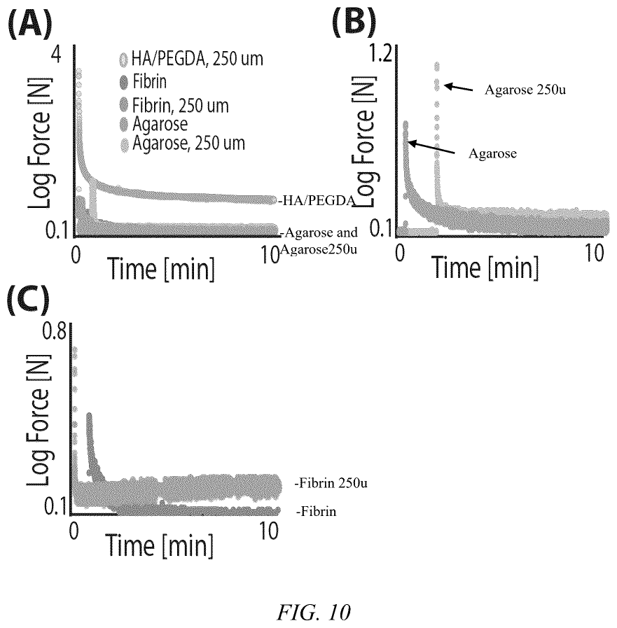

A technique has been created that is able to be mechanically tuned to a desired stiffness (FIG. 12). While it was hypothesized that smaller particles and higher viscosity gels would lead to enhanced mechanical properties, the results showed a different trend. Depending upon the resin type, there appears to be a threshold, or optimal range, where the gel can be evenly space between particles to create a solid scaffold, while also allowing the microparticles to interact and dictate the bulk mechanical properties. In the case of cartilage microparticles in the HA/PEGDA gel, the 1% gel with 250 μm sized particles led to the highest compressive modulus of 140 kPa. Each resin used in this technology can be analyzed using the same testing method (see e.g. FIG. 11), but the desired mechanical properties for the target tissue type can be adjusted by changing the viscosity of the resin the size of the microparticles, and the packing density of particles in the gel.

Finally, initial cell culture results show that when cells are introduced to the engineered microparticle construct, they migrate into, and populate the microparticles (FIG. 12). Interaction of the cells with the particles means that cells are able to attach to tissue specific attachment sites, likely initiating a signaling cascade from the ECM to the cell to proliferate and differentiate into the proper cell type. Differentiation of cells into a tissue specific lineage is a critical component of tissue regeneration. The specific migration and attachment process of cells introduced to the gel resin can be optimized through testing.

### Example 2—Engineered Microparticle Construct for Particulated and Reconstituted Cartilage for Cartilage Replacement and Repair—Materials and Methods

Tissue particulation: Tissues harvested from animal or human sources are first devitalized (flash frozen). Particulation is accomplished using a variety of methods, including mortar and pestle, or a liquid nitrogen freezer mill. Particulated tissues are size sorted using sieves in standard sizes, e.g. 0-60 microns, 60-120 microns, 120-250 microns, 250-500 microns, 500-1000 microns, and greater than 1000 microns. Particles are decellularized using standard detergents with or without subsequent DNA removal.

Collagen gel resin: Oligomeric collagen is derived and sterilized as previously described. [J. L. Bailey, et al., Biopolymers 2011, 95, 77.] This formulation was standardized based upon purity and polymerization potential as described in the ASTM standard guidance document F3089-14. [American Society for Testing and Materials, Standard Guide for Characterization of Type I Collagen as Starting Material for Surgical Implants and Substrates for Tissue Engineered Medical Products, ASTM Standard #F3089-14, 2008.]. Collagen was neutralized and polymerized at 5 mg mL-1 in specific molds (10×5×14 mm, w×t×h) allowing for densification, confocal microscopy visualization, and AFM analysis.

HA/PEGDA gel resin: Twenty-five percent thiolated HA is lyophilzed to form a spongy material. Gels are formed using a PEGDA cross linker with a ratio of 1:0.8 thiols:PEGDA. PEGDA and HA are weighed out and dissolved in PBS. The two solutions are combined with a final ratio of HA 10 mg/ml and PEGDA 8.6 mg/ml in 1% gels, or HA 20 mg/ml and PEGDA 17.2 mg/ml in 2% gels. It is envisioned that gels can be employed in the range of about 0.5% to about 3% and preferably from about 1% to about 2%.

Agarose gel resin: Agarose is formed from a low-melt variety in standard formulations (e.g. 1%, 2%, 4% or 8%). Agarose is first mixed and heated to temperatures slightly above body temperature (e.g. greater than 37 degrees C.), mixed with other constituents, and allowed to cool and polymerize.

Composite gels with microparticles and resin: Resin is created in a manner specific to the type of resin. For collagen and HA/PEGDA, protocol is described above. Microparticles are weighed out at a ratio of 0.22 g/L of resin and placed in a custom culture dish made out of PDMS with a glass slide on the bottom. Resin is dripped onto the microparticles in a cold room to ensure resin percolates fully into the microparticles. A glass slide is applied on the top to evenly distribute the gel and ensure a flat surface for mechanical testing. Composite resin and microparticles are placed at 37° C. for 30 min to facilitate Michael addition crosslinking of the resin into a gel.

Mechanical Testing: Unconfined Compression tests were performed on a Bose ElectroForce. 5500 mechanical testing system. Contact with flat construct surface was ensured using a 0.05N pre-load. Gel was compressed with a displacement of 20% of the height in 50 ms and then held at 20% displacement for 30 minutes. Instantaneous stress was determined by taking the peak force value divided by the area of the construct. Equilibrium stress was calculated by averaging the stress values for the last 1.5 minutes of the relaxation period and dividing by the area. Dividing stress values by the strain resulted in the Young's Modulus of the constructs in each condition.

Percolation Threshold: HA/PEGDA constructs were made as previously described using several ratios of resin to particles. After allowing the construct to polymerize for 30 minutes at 37° C., gels were put in 5 mL of PBS, lightly stirred, and evaluated to see if particles from the construct came loose (FIG. 4).

Construct and Cartilage Plug Staining and Imaging: Constructs were first labeled with DAPI to tag all cell nuclei and nuclear fragments that remains in the decellularized particles. 500 μL of DAPI titre was added to each sample and left to sit for 5 minutes in darkness. Gels were then washed twice with PBS. Ghost die 710 (tonbo biosciences), a cell membrane stain, was used to visualize cell phenotype and living vs. dead cells. After constructs were washed twice with PBS, 800 μL of ghost die titre was added to each construct. The samples sat at 4° C. for 30 minutes in complete darkness. After staining, constructs were washed twice with FACS buffer and fixed with 4% PFA to retain the stain for long term imaging. Constructs were then imaged using the 640 nm wavelength on a confocal microscope under a 10× objective lens (for FIG. 3). After a one-week culture period with primary chondrocytes, gels underwent the same imaging protocol but were imaged using 155 μm thick z-stacks under a 20× objective lens (FIG. 12).

Postmortem Ovine Defect Repair: Sheep legs a few hours postmortem was used to test the ability of a microparticle-gel construct to fill a cartilage defect. Ovine joint was opened to expose the medial condyle of the knee. An 8 mm plug bore created a defect. After removing the defect, cartilage microparticles were applied to fill the defect. Next, 1% HA/PEGDA gel was administered to the defect and filled in the cracks between particles. The gel in the joint was left to polymerize at room temperature naturally and achieved polymerization within 15 minutes.

### Example 3—Engineered Microparticle Construct for Particulated and Reconstituted Cartilage for Cartilage Replacement and Repair—Application

Soft tissue trauma to articular cartilage often results in degradation of joints, detrimental loss of ability to perform tasks and mobility, and increased pain and associated healthcare costs. The present invention employs decellularized and particulated tissues that promote joint preservation and restoration as a preferred option to joint loss or replacement, which will benefit orthopaedic injuries sustained by military personnel and the public at large.

The present disclosure demonstrates the percolation limits of decellularized microparticles in tunable HA/PEGDA gels to facilitate an understanding of how construct architecture influences cell signaling and mechanical integrity, and to advance new treatment options for cartilage defects that otherwise ultimately progress to OA.

There is currently no suitable cartilage tissue repair method following local trauma to cartilage, or methods to halt or prevent progress toward OA. Existing repair technologies for cartilage include the use of viable, particulated tissues that are typically derived from young human donors and require the use of tissue adhesives like fibrin. While initial reports suggest such methodologies may have some promise, there are significant challenges that still remain, including limited donor availability, limited shelf life, and the possibility for disease transmission. The technology taught herein, including the employment of decellularized, particulated tissues, provides several key advantages over existing technologies. These advantages include (1) the use of native extracellular matrix that can be derived from xenogenic, allogenic, autogenic, or syngeneic sources, (2) the ability to incorporate autologous cells from the patient that infiltrate naturally into the reconstituted matrix from underlying bone marrow, and (3) the inclusion of composites made from pro-chondrogenic matrices (e.g. hyaluronan) that allow for a moldable, scalable, and mechanically tunable final tissue construct suitable for treatment of a wide variety of orthopedic trauma presented in the clinic. In addition, the present studies of construct efficacy take advantage of new optical clearing [Calve, S., A. Ready, C. Huppenbauer, R. Main, and C. P. Neu, Optical clearing in dense connective tissues to visualize cellular connectivity in situ. PLOS One, 2015. 10(1): p. e0116662] and imaging methods [Henderson, J. T., G. Shannon, A. I. Veress, and C. P. Neu, Direct measurement of intranuclear strain distributions and RNA synthesis in single cells embedded within native tissue. Biophys J, 2013. 105(10): p. 2252-61] to visualize matrix and intracellular markers, and subcellular biomechanics, deep within the tissue. With these developments, the structural and biochemical regulation of chondrogenesis, cell signaling, and biophysics can be optimized, while also optimizing the engineered microparticle construct for cartilage suitable for in vivo implantation.

hMSCs in contact with microparticles express chondrogenic markers. While not wishing to be bound by a theory, we reasoned that dense packing in the engineered microparticle construct, combined with growth factors (e.g. TGF-β, IGF, NGF, BMP), increases chondrogenesis. In addition, close packing of particles improves structural support and mechanical properties. This allows for tuning of particle sizes and packing to best provide an implantable scaffold suitable for in vivo transplantation. hMSC-laden microparticle-HA composites can be fabricated by varying three primary factors (microparticle size, density of microparticles in HA/PEGDA, and growth factor supplementation) in an in vitro model of cartilage defect repair. Standardized hydrogel formulations (with 25% thiolated HA, 1% or 2% w/v) can be used. Multiscale function of constructs can be determined using mechanical testing in unconfined compression to test the extent that architecture and chemical stimulation affect the structural response. In parallel time course studies (i.e. cultures of 1, 2, and 3 weeks), multiscale strain can be quantified in a model of cartilage defect repair using deformation microscopy [Henderson, J. T., et al., Biophys J, 2013. 105(10): p. 2252-61]. Cell/nuclear architecture can be visualized by histology and optical clearing methods paired with immunofluorescence staining for matrix components (e.g. types II, VI, and X collagen) [Calve, S., et al., PLOS One, 2015. 10(1): p. e0116662]. Expression of mechanosensitive genes (e.g. lubricin [Neu, C. P., et. al., Arthritis Rheum, 2007. 56(11): p. 3706-14]) can be quantified by RT-PCR [Novak, T., et al., Adv Funct Mater, 2016. 26(30): p. 5427-5436], in addition to chondrogenic gene expression markers (SOX9, aggrecan, COL II, COL X). Multifactor statistics can relate and colocalize marker and strain measures, and test how microparticle-HA/PEGDA architecture recapitulates cell signaling and mechanical properties.

Close packing of small particles, with a high composite (microparticle: HA) ratio, should maximize the chondrogenic response of cells in the engineered microparticle construct for cartilage repair. The engineered construct with hyaluronan will provide a higher chondrogenic response of hMSCs compared to type I collagen-based scaffolds. Factors such as microparticle size and density can be tuned to promote mechanical integrity, scaffold architecture, and new ECM production to best mimic native cartilage, and their relation to chondrogenesis.

The in vivo utility of engineered microparticle construct for the repair of large focal defects can be established in a goat model. Studies detailed herein found that the engineered microparticle constructs exhibit exciting properties in vitro, which will translate into in vivo applications. The engineered microparticle construct will restore the functional outcomes in an in vivo model of defect repair to levels observed in native tissues and will protect the joint from degeneration following trauma.

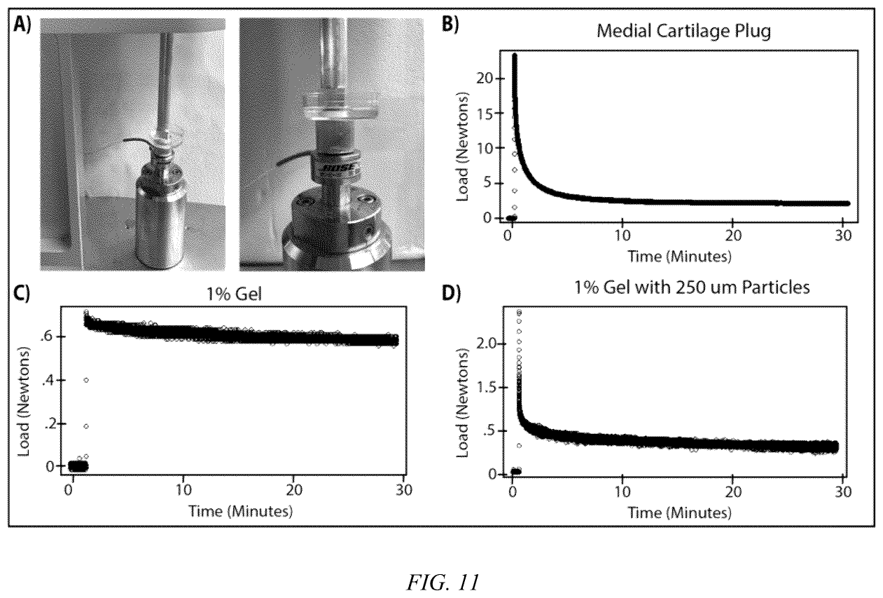

The influence of full thickness defects and the engineered microparticle construct for cartilage repair on cartilage biomechanics in an established in vivo caprine (goat) model (FIG. 13) with treatment groups: control (sham operated), microfracture as a standard of care, and graft repair using the engineered microparticle construct has been established. Morphometric MRI and biochemical (serum and synovial fluid) biomarkers (e.g. IL-1B and IL-6) can assess function [Novak, T., et al., In Vivo Cellular Infiltration and Remodeling in a Decellularized Ovine Osteochondral Allograft. Tissue Eng Part A, 20162]. Predictive statistics can be used to compare in vivo time course functional data (at 26 and 52 weeks) to direct (“gold standard”) measures of cartilage structure by histochemical grading and biomechanical testing. Validation studies in phantom and cadaveric tissues will confirm the reproducibility of assays for in vivo time course studies.

The engineered microparticle construct will restore the functional outcomes in an in vivo model of defect repair to levels observed in native tissues and will integrate with the surrounding tissue of the defect. The time course of healing in terms of architecture and biomechanics of repair and native tissues can also be defined using the in vivo model.

### Example 4—Application of Particulated and Reconstituted Tissues for Spinal Cord Regeneration

A unique regenerative bioscaffold, based on native extracellular matrix (ECM), to improve the cellular environment of the spinal cord and mitigate the secondary chemical effects leading to the formation of the cystic cavity and glial scar. Spinal cord injury (SCI) is a life-altering event for the service member, the family member, and military team. While improvements to military safety have increase personnel protection during missions, this has also led to increased survival after severe trauma including spinal cord injuries. With estimates of occurrence between 7.4% to 38%, depending on deployment location and military branch, clinical intervention and rehabilitation post-injury now require increased need for regenerative medicine for survivors. After SCI, the secondary chemical cascades involving inflammation and cellular death disrupt the microenvironment causing inhibition of neurogenesis and formation of cystic cavitations. Unfortunately, there are few effective therapies that promote and direct proper cellular growth after injury, and there is a need for strategies that exploit native ECM of the spinal cord to promote repair, and a need to determine the translational efficacy of new therapies in vivo.

New repair strategies for damaged tissue have been developed that combine decellularized and particulated tissues in a natural ECM to encourage neurite direction and growth. In pilot studies, we have shown that constructs formed from tissue microparticles and ECM influenced differentiation of stem cells toward lineages defined by the specific tissue type. In addition, human adipose tissue has similar mechanical and chemical properties to neural tissue, is an easily sourced material, and can be particulated to micron-size tissue pieces suitable for implantation [Mariman, E. C. and P. Wang. Cell Mol Life Sci, 2010. 67(8): p. 1277-92; Tukmachev, D., et al., Tissue Eng Part A, 2016. 22(3-4): p. 306-17]. Hyaluronic acid (HA) is a critical ECM glycosaminoglycan that interacts with cells via CD44 surface receptors to facilitate cell migration [Unterman, S. A., et al., Tissue Eng Part A, 2012. 18(23-24): p. 2497-506]. HA can be functionalized with thiol groups (following replacement of HA carboxyl groups) to react with poly (ethylene) glycol diacrylate (PEGDA) in a thiol-mediated Michael addition reaction. This cross-linking reaction modifies the linear molecule to form a stable 3D network that has tunable mechanics, porosity, and degradation rates [Eng, D., et al., Acta Biomater, 2010. 6(7): p. 2407-14]. The present invention provides methods for the improvement of cellular growth and decrease cystic cavitation formation after SCI through tunable bioscaffolds formed by spinal cord or adipose microparticles in HA-based hydrogels.

The decellularized microparticles encapsulated in a hyaluronic acid-based gel, as disclosed, provide positive chemical cues resulting in directed neurite growth and synaptic function. The injectable hydrogel-microparticle bioscaffold will: (1) encourage regenerative growth in the damaged spinal area; (2) mimic the structural and chemical support of the native environment; and (3) establish a novel bioscaffold for critical care intervention to minimize cellular apoptosis and promote neurogenesis.

PEGDA-HA hydrogels with decellularized ECM microparticles can be utilized as an injectable bioscaffold. After spinal cord injury, the secondary chemical cascades are detrimental to spinal cord function. Providing a tunable hydrogel with microparticles similar to the native spinal cord ECM will provide proper mechanical and chemical cues. Bioscaffolds can be created for optimal neurite extension and mechanics.

A three-factor design can be utilized to: (1) define the optimal ratio and size of microparticles to PEGDA-HA hydrogels; (2) characterize the composition of the hydrogel-microparticle bioscaffolds in terms of degradation, porosity, and stiffness; and (3) examine cellular growth and neurite extension with two decellularized ECM tissues: porcine spinal cord or human adipose tissue. Using dorsal root ganglion cells from embryonic rats, cells will be embedded in 3D PEGDA-HA hydrogels. The hydrogel formulations (e.g. with 25% thiolated HA) can be utilized with varying HA, PEDGA, and microparticle size and concentrations for optimal neural growth. Mechanical properties can be measured in compression and shear using macro- and micro-scale testing [Novak, T., et al., Adv Funct Mater, 2016. 26(16): p. 2617-2628]. Primary biological readouts for this design can include cell viability and neurite growth over the time course of weeks. Neurite growth can be measured by confocal imaging of neural cytoskeletal and nuclei markers, and viability will be assessed by live/dead staining. To mimic the common spinal fractures sustained by the military personnel, a custom impact device will provide controlled injury to the culture. Pre-polymerized hydrogel-microparticle bioscaffolds can be added to the confined injured area to allow for encapsulation of the damage area. Neurite growth and synaptic activity can be measured post injury for up to four weeks.

These studies will highlight the factors (e.g. of microparticle size, type, and density) that promote mechanical integrity, scaffold architecture, and new ECM production to best mimic the native cellular environment for proper neurogenesis.

The injectable bioscaffold can be used in an in vivo spinal cord injury model. Pilot studies demonstrated that microparticles exhibit exciting properties in vitro, suggesting successful translation in vivo. The hydrogel-microparticle bioscaffolds will improve functional outcomes in an in vivo model of defect repair and protect the spinal cord from cystic cavitations following trauma.

Adult rats will receive a hemi-section of the vertebra to mimic spinal injury. The pre-polymerized hydrogel-microparticle bioscaffold will be isotonically balanced and injected into the defect area. Decellularized microparticles from both porcine spinal column and human adipose tissue will be assessed in vivo with hydrogel composition and hydrogel-to-microparticle ratio. Biocompatibility, inflammation, and cellular engraftment will be assessed at 2, 4, and 8 weeks, consistent with the time course of loss of function [Tukmachev, D., et al., Tissue Eng Part A, 2016. 22(3-4): p. 306-17] observed in animals. Immunohistology and optical clearing methods [Calve, S., et al., PLOS One, 2015. 10(1): p. e0116662; Neu, C. P., et al., Osteoarthritis Cartilage, 2015. 23(3): p. 405-13], can be used to assess the type of cells and neurite growth within the hydrogel-microparticle scaffold and cystic cavititations (tissue volume loss) through hematoxylin-eosin staining and immunolabeling. Rats will be treated with immunosupressants for decreased graft rejection. In addition, the rats will receive integrated stress response inhibitor which crosses the blood-brain barrier to help minimize the cellular stress response. The ECM used to create the microparticles provides a novel option in providing positive feedback chemical cues to stressed cells from ECM proteins similar to the native healthy environment.

### Example 5—Engineered Microparticle Construct for Particulated and Reconstituted Tissues for Skin Replacement and Repair—Overview

The most common technique to treat severe skin defects, such as deep wounds or severe burns, consists of an autologous graft of epidermis and dermis harvested from a healthy region of a patient. This technique presents various drawbacks such as limitation of the surface area that can be treated, extended scarring and pain, or complications with infection at the healthy sites, as well as limited healing success. Here we show that decellularized extracellular matrix (d-ECM) of porcine skin samples encapsulated in a hyaluronic acid-based hydrogel provides a platform to recapitulate the mechanical environment of skin tissue, while also providing proper dermal attachment sites and growth factor reservoirs for host cells. A porcine skin when particulated and packed tightly into a gel resin, as taught herein, can mimic mechanical properties of the native skin, suggesting percolation of micronized tissues is critical to restore tissue mechanical function. Encapsulated cells remained viable after culture in d-ECM composite materials. The technique taught herein provides an acellular repair strategy that can be applied to large tissue area damage from trauma or severe burns. This simple acellular repair technology allows for rapid, point-of-care application after injury, and therefore can harness the initial repair response naturally induced in the body.

The current clinical gold standard to treat full-thickness injuries is split-thickness autologous skin grafting. Undamaged skin (epidermis and superficial part of the dermis) is extracted from a donor site on the patient and is grafted on the full-thickness wound area. The capillaries of the graft will then connect with the existing capillary network of the wound site, providing nutrients for graft survival. At the same time, the donor site will heal through re-epithelialization. The healing time in the wound site is decreased by increasing the thickness of the undamaged skin collected. However, this also leads to extended scarring and a longer recovery of the donor site, and therefore there is an important balance, and distinct size limitation on the effectiveness of this treatment. Extensive wounds covering a large region (such as heavily burned patients) cannot be treated with existing grafting techniques. Furthermore, the number of skin extractions per healthy donor site is limited, and the donor site must also heal through re-epithelialization. The loss of epidermal barrier in two locations, and the reduced immunity due to two healing sites can lead to bacterial sepsis, a commonly fatal condition. The development of new techniques for replacing large full thickness injuries would be particularly helpful in cases where options for skin graft harvesting are limited or extensive removal of grafted skin is a significant risk to the patient. A new product for extensive dermal repair is provided using a technique shown to be effective for repair of articular cartilage in-vitro. The skin product creates an acellular scaffold-based skin substitute composed of dermal microparticles encapsulated in a hydrogel, that will stimulate tissue regeneration through interaction with host autologous stem cells.

### Example 6—Engineered Microparticle Construct for Particulated and Reconstituted Tissues for Skin Replacement and Repair—Materials and Methods

Decellularized skin microparticle isolation: All skin tissue used was sourced from market weight porcine tissue within 48 hours of slaughter. Harvested tissue was frozen at −80° C. until further processing. Dermis was removed from the subcutaneous layer by pulling the tissue into tight tension and scraping off subcutaneous layer with a scalpel. Dermis tissue was pulverized using a liquid nitrogen magnetic freezer mill as previously described [T. Novak, et al. Adv. Funct. Mater. 26, 5427-5436(2016)]. Particles were sorted via a micro sieve stack to isolate for particles that were smaller than 710 μm in size (Electron Microscopy Sciences, Hatfield PA). Sorted skin microparticles were decellularized in 2% SDS for 30 hours at 37° C., and in 0.1% DNase for 4 hours to remove cellular and genetic material. Skin particles were then rinsed in PBS 5× over a 12-hour period, flash frozen in liquid nitrogen, and lyophilized.

Formation of dense hyaluronic Acid/PEGDA gel with tissue microparticles: 25% thiolated HA is lyophilized and dissolves easily when introduced to media. Gels are formed using a PEGDA cross linker with a ratio of, in one embodiment, 1:0.8 thiols: PEGDA. The two aqueous solutions are combined with a final ratio of HA 10 mg/ml and PEGDA 8.6 mg/ml. Microparticles are placed in a custom culture dish made from PDMS with a glass slide on the bottom. Resin is dripped onto the microparticles in a cold room to ensure resin percolates fully into the microparticles. A glass slide is applied on the top to evenly distribute the gel and ensure a flat surface for mechanical testing. Composite resin and microparticles are placed at 37° C. for 30 min to facilitate Michael addition crosslinking of the diacrylate groups on the PEGDA with the thiol groups on the HA molecules to form a stable 3D structure. To create denser gels, particle concentration is increased.

Confocal Imaging: Inert engineered microparticle construct skin gels are washed twice with PBS, stained for 10 minutes with a standard DAPI stain that stains the ECM of the particles, and rinsed. The gels are then imaged on an inverted Nikon Confocal microscope using a standard 405 nm laser at a 10× objective. Using ImageJ software, the ratio of particle area to gel area is measured. Each gel is imaged at 3 unique locations (in x, y, and z), and the particle: gel area fraction is averaged between the three locations.

Area ratio calculation: Engineered microparticle construct skin gels are stained in a DAPI stain for 15 minutes at a concentration of 5 μl/mL. On an inverted confocal microscope, images are acquired at 10× magnification. Using ImageJ software, a threshold is set to the image to highlight the particle portions of the image and not highlight the gel. The thresholding can be transformed into an outline and calculate the area of the particles combined, divided by the area of the whole image. For each gel, this area ratio is calculated in three separate locations and averaged to determine the area ratio of the gel.

Mechanical Testing: Unconfined Compression tests were performed on a Bose ElectroForce 5500 mechanical tester. Contact with flat gel surface was ensured using a 0.1 N pre-load. Gel was compressed with a displacement of 40% of the height at a rate 0.1%/sec to avoid effects of water stiffening in the hydrogel (rate determined from earlier experiments). Equilibrium modulus was calculated using the slope from 30% to 40% of the stress/strain curve (linear portion of the curve).

Swelling properties of polymerized gel: After polymerization, the height was measured using a BOSE Electroforce 5500 by bringing the platen to a 0.05 N Pre-load. Diameter is also measured using a caliper. Gels are then immersed in PBS for 12 hours at 37° C. Height and diameter are then calculated again to determine the radial and vertical swelling percentages.

Raman spectroscopy: Raman spectroscopy was performed by focusing a monochromatic red laser beam at a point of interest in testing (either tissue particle or gel). Most photons will interact elastically with the sample, but a small amount of light will scatter inelastically due to molecular vibrations in the sample. In this interaction with the sample, the photons either gain or lose energy and change frequency, which is collected and plotted against intensity. Raman spectra from 600 nm-1800 nm wavelengths can be collected.

Mice skin fibroblast isolation: Skin is harvested from the chest of the mouse, where the dermis is the main skin layer. The dermis is dehaired, removed from the mouse using a scalpel, cut into small pieces, cleaned of residual hair, and put in a DMEM/F12 and collagenase P digestion medium for 30 minutes under agitation at 37° C. Skin cell suspension and excess tissue particles are plated on tissue culture plastic to allow the cells to crawl out of the skin pieces and attach to the culture plate. Cells are cultured with a modified DMEM/F12 medium and passaged when 80% confluent.

CFSE staining: Cell pellet is resuspended in a 5 μm carboxyfluorescein succinimidyl ester dye solution. Cells are incubated in the stain for 20 minutes at 37° C., and stain is then deactivated using a complete medium at 37° C. for 5 minutes to quench any dye remaining in solution. During the staining period, dye diffuses easily into cells and binds covalently to all free amines creating a stable, long lasting fluorescent dye.

Evaluation of cellularized skin constructs: Engineered microparticle gels with encapsulated CFSE stained mouse fibroblasts are imaged using an inverted Nikon confocal microscope. First, gels are stained with ethidium homodimer-1 to stain for dead cells. Gels are rinsed twice in PBS, suspended in 1 ul ethidium homodimer-1/1 mL PBS suspension for 30 minutes in a standard incubator. Gels are then rinsed with PBS and put on a sterile imaging dish. Live gels are imaged using 488 nm and 561 nm lasers to view dead (red) chondrocytes to test for cell viability and cell location periodically over the 2-week culture period.

### Example 7—Engineered Microparticle Construct for Particulated and Reconstituted Tissues for Skin Replacement and Repair—Results

The engineered microparticle skin construct provides a viable option for the generation of recapitulated d-ECM. It is composed of decellularized microparticles, which are microscopic fragments of native ECM from the dermis of a xenogenic or autogenic source, embedded in hyaluronic acid. Synthetic hydrogel-based techniques have been utilized to replicate the mechanical environment of the native ECM found in dermal tissues. However, providing a supportive mechanical environment is just one function of the native ECM. Artificial materials are unable to exactly replicate the complexity of the ECM structure. These matrices are often composed of unnatural chemical components and therefore do not promote adequate differentiation of host stem cells into fibroblasts or keratinocytes. Contrasting these techniques, the use of dermal tissue to derive embedded microparticles, rather than a synthetic alternative, allows the engineered microparticle skin construct to create the ideal biochemical environment for nearby cells. The presence of native decellularized microparticles in the construct means that extracellular matrix proteins and growth factors found in a patient's own ECM will be abundant in the scaffold, providing the biological molecules important for cellular signaling events in the ECM. Because they're sourced from dermal ECM, the microparticles inherently contain skin specific attachment sites for host cells to migrate towards and attach to, promoting a regenerative repair response. The engineered microparticle skin construct is a composite of two elements: acellular microparticles of porcine dermis and a hydrogel made of hyaluronic acid (HA) and polyethylene glycol diacrylate (PEGDA). Embedding dermal microparticles in hyaluronic acid, a protein-based gel, allows the engineered construct to replicate both the mechanical and biochemical environment of the dermal ECM. HA is used as the hydrogel base because this material has been shown to be an important factor in human tissue development. Therefore, HA should support and stimulate dermal regeneration. The hydrogel naturally polymerizes at 37° C. in 30 minutes and is therefore easily able to adapt to any wound shape, while also creating a stable gel quickly once applied to the injury site.

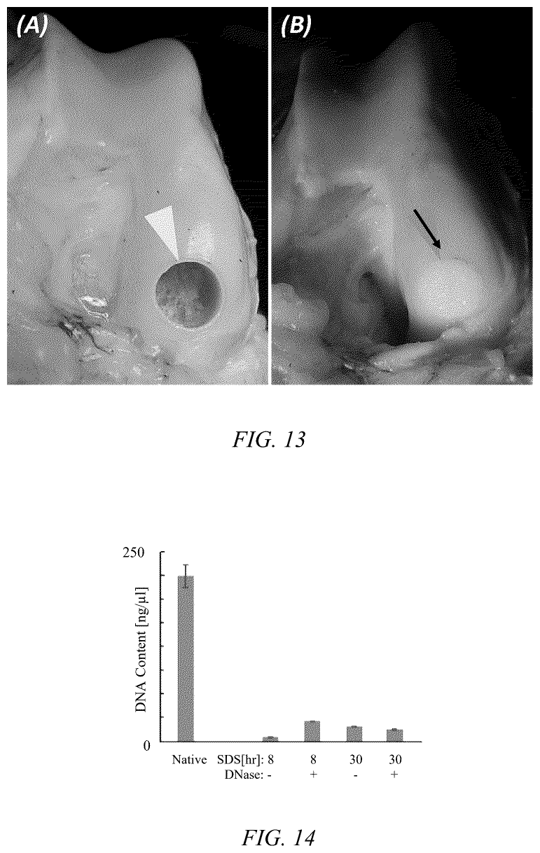

Decellularization does not remove key collagen protein from skin tissue. While the compressive modulus decreased in gels that were decellularized, raman spectroscopy showed that skin maintained many key collagenous amino acids (FIG. 15). Therefore, while the long decellularization procedure weakened the structure in some ways, the collagen proteins did not denature, and therefore the tissue still has a defined 3D structure with skin specific composition.

Increased tissue particle packing led to improved compressive mechanical properties. An increase in particle packing led to small increases in the compressive modulus of the skin mimic (FIG. 15). While the modulus in the gels does not reach that of native tissue (˜600 kPa), the highest density (0.84 Area Ratio) is ˜200 kPa, and a decellularized skin tissue that is not particulated is ˜300 kPa. This shows that the pulverization of tissue into particles does not affect the mechanics as much as the process of decellularization. With applied densification along with the high area ratio, one could very closely mimic the properties of decellularized skin to make a mechanically relevant skin scaffold.

Skin tissue swells extensively when introduced to PBS after polymerization. When introduced to PBS, gels of all particle concentrations increased their width by ˜200% and height by ˜150% (FIG. 15). This result means that even gels that have 15 mg of particles will swell about the same amount as gels that have 35 mg of particles. Because the HA/PEGDA hydrogel does not swell in either direction, this implies that skin particles will swell until constricted by space in the gel. In other words, the amount an individual particle swells in the 11 mg gel are much more than an individual particle in the 35 mg gel, but the bulk swelling of the two is the same. The microparticle-gel platform to make engineered microparticle skin construct shows that with particulation and decellularization, skin maintains its architecture while losing mechanical strength. The decrease in compressive modulus is likely tied to the large swelling observed when these tissues are introduced to PBS. As the tissue hydrates, the structure enlarges and therefore loses its compressive strength slightly.

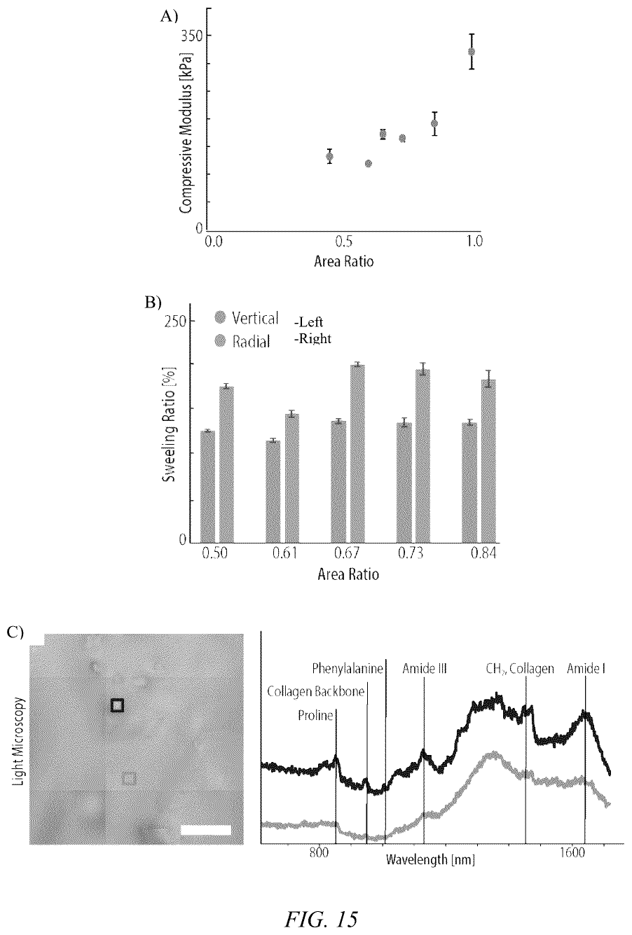

CFSE stained cells show interaction with skin tissue particles in a 3D gel and are phenotypically different than in two dimensions. When CFSE stained mouse fibroblasts are introduced into the HA/PEGDA gel surrounding dermal microparticles, they are seen on the borders of tissue microparticles (FIG. 16). Furthermore, some of the CFSE stained cells seem to be grouped right around the tissue and could possibly be inside of the decellularized tissue matrix. Skin fibroblasts display a very different phenotype when cultured on TCP for a few days, versus being encapsulated in 3D inside of an HA/PEGDA gel. The cells encapsulated in 3D are spherical, while the 2D fibroblasts have long extensions off the cell body.

One of the main components of the engineered microparticle skin construct is the microparticles encapsulated inside a hydrogel to form a dermal skin substitute. Typically, bioengineered tissue using dermal ECM, such as ALLODERM SELECT, use entire sheets of dermis. It was observed that using small particles, rather than an entire sheet, allowed tuning of the mechanical properties of the biomaterial, while still maintaining structure at the micron scale. In addition, by reducing the size of the particles increased the surface area of skin with which cells can interact, leading to a greater interaction of host cells with the tissue which increases the probability of a skin specific reaction of the cells and therefore a better chance of tissue regeneration.

The engineered microparticle skin construct has many competitive advantages over other engineered skin products. The application of the composite particle-gel system is faster and simpler than other methods. The constituents of the construct are all lyophilized and therefore stable at room temperature, which means this composite material will have a long shelf life. Furthermore, the simplicity of application of this non-cell based regenerative material will allow it to be used in emergencies where the nearest emergency room is far away. Decreasing the time between injury and repair can greatly improve outcomes for the patient. Furthermore, the engineered microparticle skin construct aims to achieve complete tissue regeneration, rather than repair, by providing the right environment for cells to initiate a regenerative signaling cascade. Therefore, the engineered microparticle skin construct could reduce time, cost, and scarring as compared to gold standard autologous skin grafts.

### Example 8—Overview-Mechanically Tunable Scaffold with Tissue Specific Signaling for Customizable Tissue Regeneration

A hallmark of native tissue is the dense extracellular matrix with high cellularity that gives rise to unique tissue-specific structure, mechanical function, and cell signaling. Unfortunately, native tissue structure is lost in damage and disease and not easily recapitulated through modern methods of tissue engineering. Moreover, while decellularized extracellular matrix (d-ECM) provides an ideal platform for regenerative medicine, large constructs do not easily recellularize, and only tissue architecture, but not cellularity, is restored.

The structure of articular cartilage is one example of the inherent biological complexity that is needed to address shortcomings in tissue regeneration. Recreating this high complexity in an engineered tissue is very difficult. The present example focuses on applying our tissue engineering technique to repair articular cartilage. The lack of blood flow and native inability of the joint to heal on its own makes articular cartilage an extreme example of the need for structure to provide proper function in regenerated tissues. Because this environment is so difficult to replicate, other strategies for regeneration have failed by either not recreating necessary mechanics or not providing proper biological signaling. An osteochondral plug is an idealized acellular tissue construct because it maintains the distinct structure of articular cartilage, and therefore similar mechanical properties. While decellularized osteochondral plugs maintain high compressive strength and a native cartilage structure, they suffer from limited diffusion of external cells and nutrients due to high density of the tissue. When decellularized plugs are used to fill a defect, there is limited cellular diffusion into the decellularized region, leading to ineffective healing and lack of integration with native tissue.

Particulate decellularized cartilage tissue can be used as a cartilage defect repair material. Some successful strategies that create a repair plug out of decellularized particles use exogenous UV crosslinking to structurally connect particles. While the matrix crosslinking technique creates an interconnected particle network, it does not create a mechanically tough material that could withstand the native cartilage environment and therefore requires extensive in-vitro culture time. It is possible to pack particles into a collagen network. Similarly to the UV based crosslinking strategies, this technique allows for cell introduction into the interparticle space, but uses a more biologically relevant protein, collagen, as the crosslinker. However, the interaction of the collagen network with cartilage cells induces an unfavorable fibrotic response, and the constructs still do not approach physiologically relevant mechanical properties. Overall, decellularized particulate cartilage solutions can promote cellularity in the inter-particle spaces but lose the organized cartilage structure and compressive strength of decellularized osteochondral allografts.

In each tissue type (i.e. skin, muscle, cartilage), there is a unique balance between structure, mechanics, and cellularity; the balance of these properties must be matched by exogenous tissue repair systems to optimize and improve regeneration. This invention establishes a tissue repair technique that can be applied to repair several tissue types. A goal of the scaffold design is to tightly pack decellularized extracellular matrix (ECM) particles, specific to the tissue needing repair, into a hyaluronic acid-based hydrogel to provide both the structural complexity and diverse molecular composition necessary for each tissue type, while also replicating the critical mechanical environment. A tissue specific environment will be able to support cellular processes critical for optimal function and therefore facilitate a regenerative response. Creating an environment that provides both biochemical and mechanical signals of the native tissue is the pathway to promoting optimal tissue regeneration.

Provided is a strategy to micronize and decellularize biological tissues and then recombine the tissue particles with cells and a support matrix to provide constructs with dense ECM and high cellularity. In connective and musculoskeletal (cartilage, skin, muscle) tissues, particles were reconstituted at controlled density with regional variation of architecture. Using articular cartilage as one model system that is well-known to be recalcitrant to repair, reconstitution of 250-micron particles (or less optimally particles within the range of 90-700 micron) that were tightly packed beyond a percolation threshold reached a compressive modulus with viscoelastic response that approached native tissue. Surprisingly, inter-particle cells repopulated dense tissue microparticles within 12 hours of construct formation, likely through chemotaxis to growth factor reservoirs, and displayed a gene expression profile similar to neocartilage. Tissue constructs can be formed into any three-dimensional shape or defect via simple injection molding or printing, and with a broad range of support materials, including matrices based on hyaluronic acid, agarose, or fibrin. This tissue engineering platform provides a unique means of dissociating and reconstituting complex biological tissues to restore dense ECM and cellularity. This platform will prove extremely useful in numerous regeneration applications because it is simple and acellular, composed of only gel and particle constituents, while also enabling recellularization, and offering tunable mechanics and formability.

### Example 9—Materials and Methods—Mechanically Tunable Scaffold with Tissue Specific Signaling for Customizable Tissue Regeneration

Cartilage Microparticle Preparation: All cartilage tissue was sourced from market weight porcine tissue (200 separate animals) within 48 hours of slaughter. Cartilage tissue was harvested as previously described [T. Novak et al., In Vivo Cellular Infiltration and Remodeling in a Decellularized Ovine Osteochondral Allograft, doi: 10.1089/ten.tea.2016.0149.]. Briefly, tissue was extracted by exposure of the knee joint space and scalpel removal of cartilage tissue (care was taken to not include calcified tissue). Harvested tissue was frozen at −80° C. until further processing. Tissue was pulverized using a liquid nitrogen magnetic freezer mill as previously described [T. Novak et al., Adv. Funct. Mater. 26, 2617-2628(2016)]. Particles were sorted via a micro sieve stack to isolate for particles that were smaller than 250 μm in size (Electron Microscopy Sciences, Hatfield PA). Sorted cartilage microparticles were decellularized in 2% SDS for 8 hours at 37° C., and in 0.1% DNase for 3 hours to remove cellular and genetic material to specifications as previously described [C. W. Cheng, et al., Biotechnol. Adv. 32, 462-484(2014)]. Cartilage particles were then rinsed in PBS 5× over a 12-hour period, flash frozen in liquid nitrogen, and lyophilized.

Skin Microparticle Preparation: All skin tissue was sourced from market weight porcine tissue within 48 hours of slaughter. Tissue was pulverized using a liquid nitrogen magnetic freezer mill as described for cartilage. The decellularization procedure was the same as for cartilage, except with 30 hours 2% SDS treatment and 750 μm particles.

Muscle Microparticle Preparation: All muscle tissue was sourced from market weight porcine tissue within 48 hours of slaughter. The procedure was the same as for cartilage, except with 24 hours 2% SDS treatment and 750 μm particles.

Formation of the Engineered Microparticle Constructs: Tissue Microparticles in a Hydrogel Support Matrix: 25% thiolated HA is lyophilized and dissolves easily when introduced to media. Gels are formed using a PEGDA cross linker with a ratio of 1:0.8 thiols: PEGDA. The two aqueous solutions are combined with a final ratio of HA 10 mg/ml and PEGDA 8.6 mg/ml. Microparticles are placed in a custom culture dish made from PDMS with a glass slide on the bottom. Resin is dripped onto the microparticles in a cold room to ensure resin percolates fully into the microparticles. A glass slide is applied on the top to evenly distribute the gel and ensure a flat surface for mechanical testing. Composite resin and microparticles are placed at 37° C. for 30 min to facilitate Michael addition crosslinking of the diacrylate groups on the PEGDA with the thiol groups on the HA molecules to form a stable 3D structure. To increase packing density, PDMS mold is placed in a centrifuge and spun at 4000 rpm for 20 minutes during centrifugation.

Confocal Imaging to Calculate Volume Fraction: The engineered microparticle construct gels are washed twice with PBS, stained for 10 minutes with a standard DAPI stain that stains the ECM of the particles, and rinsed. The gels are then imaged on an inverted Nikon Confocal microscope using a standard 405 nm laser at a 10× objective. Using ImageJ software, the ratio of particle area to gel area is measured. Each gel is imaged at 3 unique locations (in x, y, and z), and the particle: gel area fraction is averaged between the three locations.

Area Ratio Calculation: Engineered microparticle construct gels are stained in a DAPI stain for 15 minutes at a concentration of 5 μl/mL. On an inverted confocal microscope, images are acquired at 10× magnification. ImageJ software was used to threshold the image to highlight the particle portions of the image and not highlight the gel. The thresholding can be transformed into an outline and the area of the particles combined can be calculated, divided by the area of the whole image. For each gel, this area ratio is calculated in three separate locations, and averaged to determine the area ratio of the gel.

Raman Spectroscopy: Raman spectroscopy was performed by shooting a monochromatic red laser beam at a point of interest in testing (either tissue particle or gel). Most photons will interact elastically with the sample, but a small amount of light will scatter inelastically due to molecular vibrations in the sample. In this interaction with the sample, the photons either gain or lose energy and change frequency, which is collected and plotted against intensity. Raman spectra from 700 nm-1700 nm wavelengths was collected.

Mechanical Testing to Determine Percolation Threshold: Unconfined Compression tests were performed on a Bose ElectroForce 5500 mechanical testing system. Contact with flat gel surface was ensured using a 0.1 N pre-load. Gel was compressed with a displacement of 40% of the height at a rate 0.1% per second to avoid effects of water stiffening in the hydrogel (rate determined from earlier experiments). Equilibrium modulus was calculated by finding the slope from 30% to 40% of the stress/strain curve (linear portion of the curve).

Mechanical Testing at Physiological Rates of Walking: Unconfined compression testing was performed on a Bose ElectroForce 5500 mechanical testing system. Contact with gels was ensured using a 0.05N pre-load, followed by 20% compression of the gel in 50 ms. The platen was held at 20% compression for 30 minutes to evaluate equilibrium modulus (calculated by finding slope of curve at the last 10 minutes of the relaxation period.

Chondrocyte Isolation: Cartilage is extracted from bovine stifle (knee) joints from 2-week-old calves within 12 hours of slaughter. The joints were opened under aseptic conditions, exposing femoral condyles. Cartilage tissue was obtained via scalpel scraping from both lateral and medial condyles. After rinsing the tissue slices with PBS (3×), chondrocytes were isolated by digestion with 0.2% collagenase-P (Roche Pharmaceuticals, Nutley, NJ) for 6 hours. Digested cells were then washed with chondrogenic media (10% FBS, chemically defined Dulbecco's modified Eagle medium: nutrient mixture F12 supplemented with 0.1% bovine serum albumin, 100 units/mL penicillin, 100 μg/mL streptomycin, and 50 μg/mL ascorbate-2-phosphate) before staining and encapsulation.

CFSE Staining of Primary Chondrocytes: Before plating chondrocytes are stained with a green, fluorescent die using the following protocol. Cell pellet is resuspended in a 5 μm carboxyfluorescein succinimidyl ester dye solution. Cells are incubated in the stain for 20 minutes at 37° C., and stain is then deactivated using a complete medium at 37° C. for 5 minutes to quench any dye remaining in solution. During the staining period, dye diffuses into cells and binds covalently to free amine groups creating a stable, long lasting fluorescent dye.

Confocal Imaging of Cellularized Cartilage Constructs: Engineered microparticle cartilage construct gels with encapsulated CFSE stained primary chondrocytes are imaged using an inverted Nikon confocal microscope. First, gels are stained with ethidium homodimer-1 to stain for dead cells. Gels are rinsed twice in PBS, suspended in 1 μl ethidium homodimer-1/1 mL PBS suspension for 30 minutes in a standard incubator. Gels are then rinsed with PBS and put on a sterile imaging dish. Live gels are imaged using 488 nm and 561 nm lasers to view dead (red) chondrocytes to test for cell viability and cell location periodically over the 2-week culture period.

Gene Expression of Chondrocyte-laden Engineered Microparticle Cartilage Construct Gels: Total RNA isolation was performed using the E.Z.N.A Total RNA kit (Omega Tek). HA/PEGDA matrices were homogenized for 2 minutes (TissueRuptor, QIAzol lysis Reagant, Qiagen, The Netherlands) and cleaned from protein using a chloroform precipitation. Total RNA was reversed transcribed into complimentary DNA (cDNA, iScript Reverse Transcription Supermix, Bio-Rad) using a thermocycler and Quantitative Real-Time PCR (CFX96 Touch, Bio-Rad) was performed using SsoAdvanced SYBR Green Supermix and the CFX96 Touch adthermocycler (Bio-Rad, Hercules CA, USA). Several genes were investigated for chondrogenic differentiation gene expression, by comparing expression in a HA/PEGDA gel to expression in the particle filled gel. For all samples, glyceraldehyde 3-phosphate dehydrogenase (GAPDH) and B2M were utilized as the housekeeping genes. Known chondrocyte differentiation genes (SOX9, Col1A2, Col2A1, ACAN) were measured in all groups. All samples were normalized to the housekeeping genes. All primers were specific for all known isotopes and are separated by at least on intron or span an exon-exon junction, if splicing information was available.

### Example 10—Results—Mechanically Tunable Scaffold with Tissue Specific Signaling for Customizable Tissue Regeneration

The Engineered Microparticle Constructs as a Flexible and Adaptable Strategy for Tissue Repair: A general approach was developed to create tailored and engineered biomaterials using native tissues that closely mimic and recreate tissues of the body. The process involves breaking a healthy tissue into very small (micron scale) fragments or particles, chemically processing the particles, and then recombining them through the use of a support matrix, often using a hyaluronic acid-based hydrogel, to produce a biologically and mechanically similar tissue to the initial native tissue. Using the same method and gel material, with different tissue types, engineered microparticle constructs have been created for (1) cartilage, (2) skin, and (3) muscle. The reconstitution of these three tissue types using the engineered microparticle construct protocol demonstrates that this method is an adaptable, simple, tunable, and diverse method to produce composite materials to fill damaged regions in several tissue types. Through the use of the adaptable engineered microparticle construct protocol we demonstrate the production of mechanically unique tissue mimetic materials by encapsulating size sorted, decellularized microparticles from three tissue types into a mechanically tunable composite material (FIG. 1).

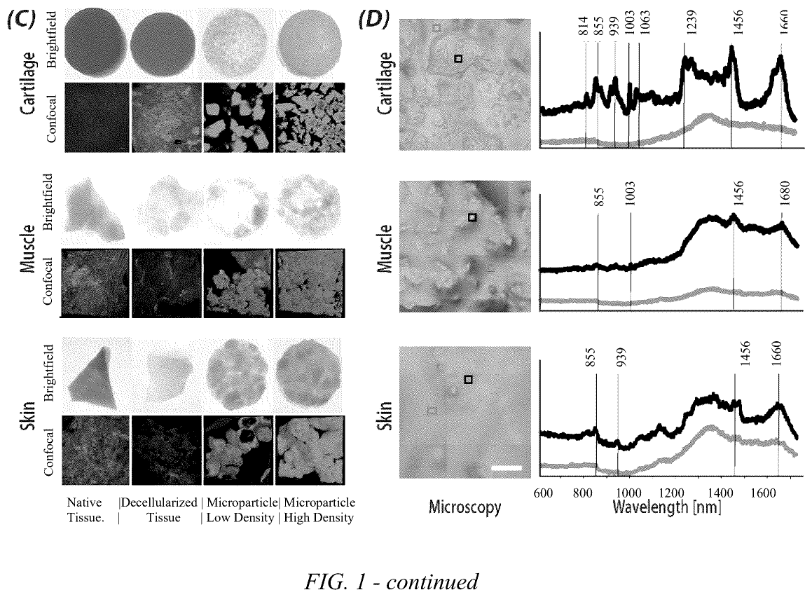

Microparticles Encapsulated in Gel Create a Tunable Mechanical Platform: The addition of cartilage microparticles into a hyaluronic acid-based hydrogel increased the modulus of the material (FIG. 2). Furthermore, the time vs. load profile of loading under physiologically relevant walking rates showed a distinct relaxation curve in particle filled gels, similar to the relaxation curve in native cartilage samples. At fast rates, the instantaneous modulus was highest in gels that had larger particles (250 μm), and a higher crosslinked gel density (2%). However, more consistency was found in 1% gels that have a lower crosslinking gel density due to the slower polymerization rate and therefore were chosen to further investigate the mechanical properties of 1% crosslinked gels containing 250 μm particles.

Conservation of Tissue Specific Composition: Raman Spectroscopy is a noninvasive technique commonly used to assess the structural composition of a substance that works by shining light of a specific wavelength that scatters when it hits certain protein macrostructures. Because collagen constitutes an important structural component of many native tissues and is responsible for much of the mechanical behavior of the ECM of native tissues, raman spectroscopy was used to test whether the signatures of native collagen structures were present in each tissue type after the process of decellularization. The raman spectra for each of the decellularized tissue samples of porcine skin, muscle, and cartilage confirmed that these tissues all have unique compositional structures, shown by the variations in the raman spectra (FIG. 1). However, the spectra from all three of these tissue types displayed many typical collagen peaks, which shows that the decellularization process, used to remove cells from the donor tissue that microparticles are being sourced from, did not extensively denature the collagen. The raman peaks identified in cartilage include C—C stretching (817), hydroxyproline (855), C—C collagen backbone (939), Phenylalanine (1003), Proline (1063), Amide III (1239), CH2CH3 confirmation collagen assignment (1456), and Amide I (1660). Muscle and skin also show peaks at a few of these typical collagen peaks (855, 1003, 1456, and 1660). This confirmation of intact collagen structure within the decellularized tissues used to produce microparticles from each tissue further supports the mechanical data, further validating how it is possible that when the engineered microparticle construct is at percolation, the decellularized microparticles filled gels are able to produce mechanical response profiles comparable to native tissue.

Percolation Threshold in Cartilage Gels: The benefits of reaching a mechanical percolation point were demonstrated by slow rate mechanical compression testing using cartilage-based microparticles as a proof of principle. Gel composites were compressed at a rate of 0.1%/second to avoid effects of liquid stiffening behavior in the hydrogel (FIG. 2). Increasing the cartilage microparticle density in HA/PEGDA hydrogels linearly increased the compressive modulus of the material until a percolation point was reached. To achieve a compressive modulus similar to native tissue, the microparticle concentration in HA/PEGDA gels was increased from a volume fraction of 0 to 0.5, increasing the equilibrium modulus 5-fold (˜10 kPa to ˜50 kPa). Initially it appeared that 0.5 units/volume was the maximum concentration of microparticles that could be solubilized in the gel samples. However, additional centrifugation was demonstrated to mechanically compress the particles in each sample during polymerization up to a volume fraction of 0.6. The increased density of microparticles from 0.5 to 0.6 achieved by centrifugation led to another 5-fold increase in the equilibrium modulus, finishing at approximately 250 kPa (FIG. 2). To assess the mechanical basis for the large compressive moduli, jump between 0.5 and 0.6 volume fraction containing gels, a percolation model was fit to this data. The general effective medium theory model was used because it is designed for composite materials where the constituent pieces are random shapes and orientations. For this data set, the material composite is a soft hydrogel with constituent decellularized cartilage microparticles. The model considers the individual moduli of the hydrogel and the cartilage to apply scaling factors to each component of the composite gel. As the concentration of particles in a gel are increased, the particles must pack more tightly together in the gel. The percolation theory predicts that as the particle packing increases the particles begin to contact each other directly to create a new network that transfers mechanical loads through the particle region of the composite, rather than the gel. This new network of direct particle contacts leads to a dramatic increase in compressive modulus that begins to approach values of the compressive modulus of the particle substance itself, or native cartilage for the data set. Based on the input of the mechanical testing of these gels, the model determined that the percolation threshold for the cartilage microparticle HA/PEGDA gels lies at 0.55 volume fraction, the microparticle concentration where there is a strong inflection point on the graph (FIG. 2).

Chondrocytes Encapsulated in the Gel Fill the Intra and Inter Particle Spaces of the Particles:

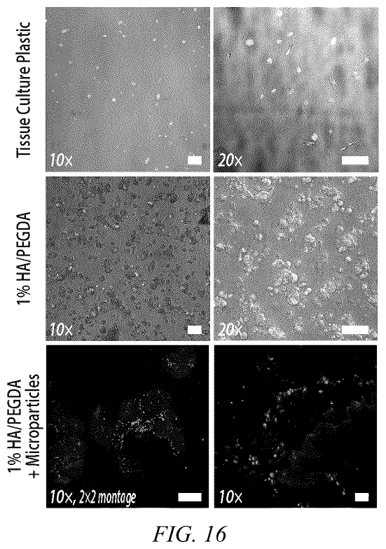

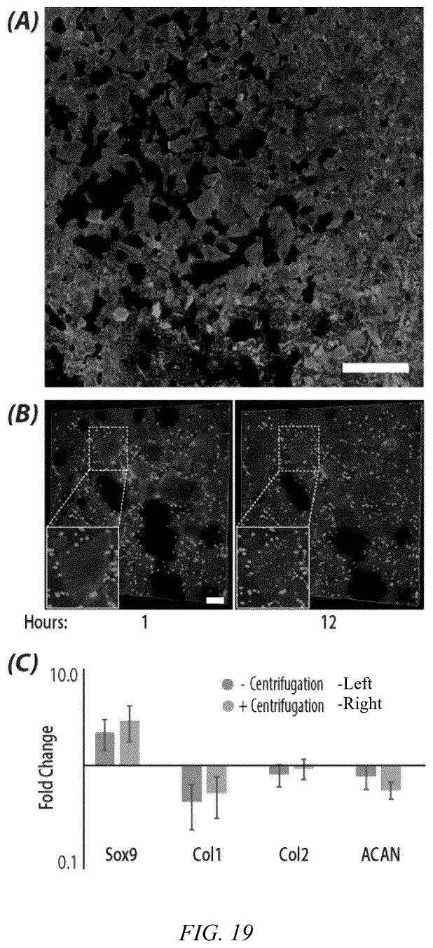

To investigate the fate of cells when introduced to the engineered microparticle construct, cartilage was again used as an example system. Primary chondrocytes were extracted from young bovine knee joints and then stained with a fluorescent proliferation die, CFSE. When the stained chondrocytes were introduced to the gel particle suspension, it was found that CFSE stained cells had recellularized the cartilage microparticles, effectively moving inside of many of the particles in each gel. By imaging the constructs each day through the 14-day culture, it was observed that cells appear in the particles within the first two days of culture (FIG. 17). Using a live imaging system for the first day of the culture, it was demonstrated in real time that within the first 12 hours, chondrocytes were re-located inside of the particles, as well as in the gel spaces around the particles (FIG. 19). Furthermore, by imaging a large region of one of these gels (˜4 mm), it was observed that this trend was global phenomenon across the gel and not restricted to any unique gel region or specific unique particles (FIG. 19). Finally, gene expression data derived from extracting RNA from the embedded chondrocytes followed by RT-qPCR showed that cells in percolated particle gels upregulated key proteins such as Sox9 and Collagen II, compared to chondrocytes in the simple 3D hydrogel without particles packed to percolation. This data demonstrates that the introduction of primary chondrocytes to engineered microparticle cartilage construct at the percolation threshold leads to encouraging cellular movement and gene expression data; suggesting that the cartilage construct provides a favorable environment to facilitate the normal cell behavior and proliferation of chondrocytes.

A strategy has been defined to micronize and decellularize biological tissues and then recombine the tissue particles with cells and a support matrix to provide the engineered microparticle constructs, i.e. constructs with dense ECM and high cellularity. The constructs can be formed with tissue-specific architectures and structural characteristics, tunable shape and mechanical properties, and with recellularized intra-construct particles. In one specific application, decellularized cartilage microparticles in a hyaluronic acid-based hydrogel created a tunable, stiff, and chondrogenic matrix. While the compressive modulus of the construct was lower than native cartilage that underwent the same processing and mechanical testing protocol (˜800 kPa), the increase in the volume fraction of decellularized particles improved stiffness dramatically, increased numbers of cell-matrix interactions, and improved chondrocyte gene expression.

Percolation theory is a mathematical model that has been applied in many mathematic, scientific, and engineering disciplines, to explain common natural phenomena that involve multiphase materials. Percolation theory in materials originally began as a way to mathematically model the mechanical behavior of identical objects that are either randomly or uniformly distributed through a medium. A common difficulty in applying percolation theory to biological applications is that often biological materials are not made of identical objects (shape, composition, etc.). Recently, an adaptation of previous percolation models bridged percolation and homogenization theories to create the General Effective Medium theory (GEM), which models continuum mechanics of random multiphase materials. Over a period of compression, this model can mathematically predict the percolation threshold, which is the point at which the mechanics of the composite system are dictated by the stiffer random constituent pieces rather than the softer surrounding matrix. Based on percolation theory applied in other disciplines, tissue specific microparticles packed together at, or past, their percolation threshold will provide the necessary mechanical environment and to best recapitulate and integrate with native tissue. The packing of microparticles, derived from the ECM of native tissue to a concentration past the percolation point will yield both the necessary biochemical and biomechanical properties necessary for reconstituting a specific tissue.

The percolation threshold of the particles encapsulated within the gel is achieved through increased particle packing into our standard hyaluronic acid hydrogel base, however we have also shown capabilities to achieve this in other gel base substrates (i.e. agarose and collagen). We have shown that packing density can only increase mechanics to a certain point. Once this point is reach, centrifugation is necessary to go beyond the percolation threshold and make gels with mechanics mimicking that of native tissue. In turn, this means that cells introduced to the gel phase of the composite material at high density, contact many tissue particle surfaces. Therefore, we have developed a material that makes cell encapsulation simple while also ensuring that cells have many attachment sites to provide tissue specific signaling pathways necessary for growth and regeneration. In the engineered microparticle cartilage construct of the present invention, chondrocytes introduced to the composite localized both around and within the particles, and increased packing led to an upregulation of key chondrogenic markers in the gene expression of encapsulated cells. In previous studies using decellularized cartilage, the matrix was too dense for cells to migrate and localize within the cartilage ECM. Therefore, the localization within the particles in composite gels at their percolation threshold is both surprising and encouraging for regeneration.

The simple techniques and designs taught herein allow for high cell-tissue contact, while creating a tunable tissue scaffold that can be optimized for application area. We have created a novel composite material that is able to utilize the structure and composition of native 3D extracellular matrices, while also creating an environment that is tunable and mimics the mechanics of a native environment. This material composite is flexible so that it can be used for several specific tissue types, and tunable so that mechanics and size of the defect fill can be easily adjusted.

Furthermore, creating a gel that provides proper biochemical signaling to introduced cells, promotes a platform for cells to generate their own tissue specific proteins and basement membrane. In many tissue engineered solutions, integration between the engineered fill and the native surrounding tissue is very difficult. Our composite material will match the mechanics and biochemical composition of the surrounding tissue and therefore will promote cells both in the defect fill and in the surrounding tissue to interact, forming a promising platform for cell communication and integration.

The flexibility of the engineered microparticle construct is shown herein using porcine cartilage, skin, and muscle. However, the source of the native tissue is independent of the scaffold design, and therefore can be: xenogenic, allogenic, autogenic, syngeneic. The tissue can be harvested from any tissue that is to be regenerated, e.g. cartilage, skin, ligament, meniscus, tendon, muscle, heart, brain, lung, etc. Once collected, the tissue can be pulverized to any size on the micron to millimeter scale, decellularized, and can then be encapsulated in the engineered microparticle construct platform. The resin used for the platform can be anything that begins in a fluid form and hardens with body temperature (e.g. agarose, fibrin, collagen, PLA, HA, PEGDA/HA, fibrin glue, etc.). The platform design creates packable, densifiable tissue repair material with mechanical rigor to withstand in vivo loading. Due to the heat polymerizable nature of the resin, the design is such that the composite can encapsulate xenogenic, allogenic, autogenic, syngeneic cell sources that are primary, stem, progenitor, engineered, or altered/transformed/immortalized.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

1-36. (canceled)

37. A method of producing a composite biomaterial for tissue regenerative medicine comprising the steps of:
providing a tissue sample;
devitalizing the tissue sample;
particulating the devitalized tissue;
size-sorting the particulated tissue within the size range of 60 micrometers to 700 micrometers to yield particulated tissue of random size and shape within the defined range;
amorphous packing the size-sorted tissue microparticles within a resin composition at or beyond a percolation threshold having a volume ratio of at least 0.57; and
polymerizing the packed tissue microparticles to form a stable composite biomaterial.

38. The method of producing a composite biomaterial for tissue regenerative medicine according to claim 37 wherein the composite biomaterial is polymerized within a mold, a defect tissue void, or via additive manufacturing.

39. The method of producing a composite biomaterial for tissue regenerative medicine according to claim 37 wherein the resin is a HA/PEGDA gel resin of about 15-30% thiolated HA with about 0.5% to about 3% w/v HA/PEGDA

40. The method of producing a composite biomaterial for tissue regenerative medicine according to claim 37 wherein the tissue for the tissue microparticles is a tissue selected from the group consisting of spinal cord tissue, adipose tissue, skin tissue, cartilage, ligament, meniscus, tendon, muscle, heart, brain, and lung tissue.

41. The method of producing a composite biomaterial for tissue regenerative medicine to claim 37 wherein the particulated tissue sample is size sorted to a size from about 60 μm to about 500 μm.

42. The method of producing a composite biomaterial for tissue regenerative medicine according to claim 37 wherein a form or mold is used in the polymerization step to produce a polymerized microparticle-resin composite having a defined shape.

43. The method of producing a composite biomaterial for tissue regenerative medicine according to claim 37 wherein the defined shape matches or approximates a tissue void to be filled in a subject.

44. The method of producing a composite biomaterial for tissue regenerative medicine according to claim 48 further comprising the step of adding one or more soluble factors to form a suspension within the resin.

45. The method of producing a composite biomaterial for tissue regenerative medicine according to claim 37 wherein the soluble factor is a factor selected from the list consisting of growth factors, cytokines, peptidoglycans, anti-inflammatory compounds, anti-senescent compounds, and cross-linking agents.

46. The method of producing a composite biomaterial for tissue regenerative medicine according to claim 37 wherein cells can be added before or after polymerization to promote cell infiltration or recellularization of the whole composite biomaterial.

47. A method of producing a composite biomaterial for tissue regenerative medicine comprising a gel resin of a thiolated hyaluronic acid and poly(ethylene glycol) diacrylate in combination with tissue microparticles, comprising the steps of:
thiolating hyaluronic acid such that hyaluronic acid has about 15-25% of the carboxyl groups in the hyaluronic acid converted to thiol groups;
concentrating the hyaluronic acid to be less than about 2% of the composite (weight/volume);
forming the gel resin by crosslinking the thiol groups within the thiolated hyaluronic acid with poly(ethylene glycol) diacrylate via a thiol-mediated Michael addition crosslinking reaction, at temperatures at or above 37° C. and without photocrosslinking;
packing the tissue microparticles at a density within the gel resin at or beyond a percolation threshold for multiphase materials, defined as 0.57 particle/composite ratio (area/area or volume/volume).

48. The method of producing a composite biomaterial according to claim 47 wherein the tissue microparticles are random and multiphase materials, including solid and fluid phases, and having a plurality of shape and composition.

49. The method of producing a composite biomaterial according to claim 47 wherein the gel resin comprises hyaluronic acid, fibrin, collagen, agarose or combinations thereof.

50. The method of producing a composite biomaterial according to claim 47 wherein the tissue microparticles have a plurality of sizes and shapes with a maximum diameter in the range of about 60 micrometers to about 700 micrometers.

51. The method of producing a composite biomaterial according to claim 47 wherein the tissue for the tissue microparticles is a tissue selected from the group consisting of spinal cord tissue, adipose tissue, skin tissue, cartilage, ligament, meniscus, tendon, muscle, heart, brain, and lung tissue.

52. The method of producing a composite biomaterial according to claim 47 wherein the composite biomaterial is prepared for implantation in a subject and the tissue is xenogenic, allogenic, or autogenic to the subject.

53. A method of producing a composite biomaterial according to claim 47 wherein the composite biomaterial has a defined shape that matches or approximates a tissue void to be filled in a subject.

54. A method of producing a composite biomaterial according to claim 47 wherein the composite biomaterial does not undergo crosslinking with UV light.
